(19) United States
(12) Patent Application Publication
(10) Pub. No.: US 2026/0224285 A1
(43) Pub. Date: Aug. 6, 2026

(54) SYSTEM AND METHOD FOR POSITIONING AND OPERATING OF A SURGICAL CUTTING INSTRUMENT

(71) Applicants: Smith & Nephew, Inc., Memphis, TN (US); Smith & Nephew Orthopaedics AG, Zug (CH)

(72) Inventors: Brett J. Bell, Mendon, UT (US); Shantanu Vyas, Pittsburgh, PA (US)

(73) Assignees: Smith & Nephew, Inc., Memphis, TN (US); Smith & Nephew Orthopaedics AG, Zug (CH)

(21) Appl. No.: 19/448,442

(22) Filed: Jan. 14, 2026

Related U.S. Application Data
(60) Provisional application No. 63/751,973, filed on Jan. 31, 2025.

(51) Int. Cl. A61B 34/10; A61B 17/00; A61B 17/14

(52) U.S. Cl. CPC A61B 34/10; A61B 17/14; A61B 2017/00199; A61B 2034/107

(57) ABSTRACT

A system and a method for positioning and operating of a surgical cutting instrument. Dimensions associated with an implant surface plane of an implant are received. The implant surface plane is defined by at least one transition edge separating the implant surface plane from another implant surface plane of the implant. The surgical instrument includes a cutting blade for cutting a bone surface in the bone corresponding to the implant surface plane. Cutting blade parameters including a reference distance representative of a distance from a reference location on the cutting blade to the transition edge, a normal distance from the transition edge to a surface of the cutting blade, and a direction of cutting of the cutting blade are identified. A reach parameter of the cutting blade in relation to the bone surface is determined. The cutting blade is aligned using the reach parameter.

### CROSS-REFERENCE TO RELATED APPLICATIONS

This is a non-provisional of, and claims the benefit of the filing date of, pending U.S. Provisional Patent Application No. 63/751,973, filed Jan. 31, 2025, entitled “System and Method for Positioning and Operating of a Surgical Cutting Instrument,” the entirety of which application is incorporated by reference herein.

### BACKGROUND

Robotically assisted orthopedic surgeries involve use of robotic systems to aid surgeons in performing precise and minimally invasive procedures, such as, for example, knee and hip replacements. These systems typically include robotic arms equipped with surgical instruments and high-definition cameras that provide enhanced views of the surgical area. Using these systems, surgeons are aided in determining specific surfaces to resect, thereby increasing accuracy in implant placement, reduced risk of complications, shorter recovery times, less postoperative pain, and smaller scars compared to traditional open surgeries. Further, robotically assisted allow for more consistent and predictable surgical outcomes, benefiting both patients and surgeons. Implants used in such procedures typically have complex (e.g., ruled) surface structure requiring precise cutting of bone tissue to ensure proper placement. The existing robotic surgical systems require manual selection of target surface but are unable to perform resections of ruled surfaces.

### SUMMARY

This summary is provided to introduce a selection of concepts in a simplified form that are further described below in the detailed description. This summary is not intended to identify key features or essential features of the claimed subject matter, nor is it intended as an aid in determining the scope of the claimed subject matter.

In some examples, the present disclosure relates to a method for positioning and operating a cutting instrument. The method may include receiving, using at least one processor of a surgical instrument, one or more dimensions associated with an implant surface plane of an implant. The implant surface plane may be defined by at least one transition edge separating the implant surface plane from another implant surface plane of the implant. The implant may be configured for implantation into a bone of a patient. The surgical instrument may include a cutting blade for cutting a bone surface in the bone corresponding to the implant surface plane. The method may also include identifying, using at least one processor, one or more surgical instrument limitation parameters, and one or more cutting blade parameters including a reference distance representative of a distance from a reference location on the cutting blade to at least one transition edge, a normal distance from at least one transition edge to a surface of the cutting blade, and a direction of cutting of the cutting blade. The method may further include determining, using at least one processor, a reach parameter of the cutting blade in relation to the bone surface based on one or more dimensions, one or more surgical instrument limitation parameters, and one or more cutting blade parameters. The reach parameter may indicate an ability of the cutting blade to cut the bone surface using one or more dimensions, one or more surgical instrument limitation parameters, and one or more cutting blade parameters. The method may also include performing, in accordance with the reach parameter, alignment of the cutting blade to the bone surface.

In any of the preceding or subsequent examples, performing may include automatically performing, in accordance with the reach parameter, the alignment of the cutting blade.

In any of the preceding or subsequent examples, the alignment of the cutting blade to the bone surface may be performed using at least one of: the implant surface plane, the another implant surface plane, or any combination thereof.

In any of the preceding or subsequent examples, the alignment of the cutting blade to the bone surface may be performed using a tangent line of at least one of: the implant surface plane, the another implant surface plane, or any combination thereof, wherein the bone surface is at least one of: a splined bone surface, an analytically defined surface, or any combination thereof.

In any of the preceding or subsequent examples, the method may also include actuating, using at least one processor, the cutting blade in accordance with the reach parameter.

In any of the preceding or subsequent examples, the method may include removing at least a portion in a plurality of portions of the bone surface using the alignment of cutting blade.

In any preceding or subsequent examples, the actuating, upon the reach parameter indicating an inability of the cutting blade to cut the bone surface using the implant surface plane, may include preventing cutting by the cutting blade.

In any preceding or subsequent examples, the actuating, upon the reach parameter indicating an ability of the cutting blade to cut the bone surface using the implant surface plane, may include aligning the cutting blade using the implant surface plane, selecting a side of the cutting blade proximate to the implant surface plane, and actuating the cutting blade for cutting the bone surface using the selected side.

In any preceding or subsequent examples, the actuating, upon the reach parameter indicating an ability of the cutting blade to cut the bone surface using a plurality of implant surface planes of the implant, may include identifying a first implant surface plane in the plurality of implant surface planes based on at least one of: a smallest distance representative of a distance from the reference location on the cutting blade to at least one transition edge of the first implant surface plane, a smallest rotational angle of the cutting blade, or any combination thereof, aligning the cutting blade using the first implant surface plane, selecting a side of the cutting blade proximate to the first implant surface plane, and actuating the cutting blade for cutting the bone surface using the selected side.

In any preceding or subsequent examples, the surgical instrument may be a surgical saw.

In any preceding or subsequent examples, one or more dimensions may include one or more plane coordinates associated with at least one transition edge between at least one implant surface plane and at least another implant surface plane in a plurality of implant surface planes of the implant. The reach parameter may be determined based on a combination of a minimum absolute distance between the distal end of the cutting blade and one or more coordinates associated with the transition edge and a negative normal distance, the negative normal distance is determined using a difference between the normal distance and the reference distance and the negative direction of cutting of the cutting blade.

In some examples, the current subject matter relates to a method for positioning and operating a surgical cutting instrument. The method may include receiving, using at least one processor of a surgical instrument, one or more dimensions associated with an implant surface plane of an implant. The implant surface plane may be defined by a plurality of splined implant surfaces. Each splined implant surface may include at least one transition edge separating one splined implant surface from another splined implant surface in the plurality of splined implant surfaces. The implant may be configured for implantation into a bone of a patient. The surgical instrument may include a cutting blade for cutting a bone surface in the bone corresponding to at least one splined implant surface in the plurality of splined implant surfaces. The method may include identifying, using at least one processor, one or more surgical instrument limitation parameters and one or more cutting blade parameters including a reference distance representative of a distance from a reference location on the cutting blade to at least one transition edge of at least one splined implant surface in the plurality of splined surfaces, a normal distance from at least one transition edge to a surface of the cutting blade, and a direction of cutting of the cutting blade. The reference distance may be determined based on an orthogonal projection to the implant surface plane. The method may also include determining, using at least one processor, a reach parameter of the cutting blade in relation to the bone surface based on one or more dimensions, one or more surgical instrument limitation parameters, and one or more cutting blade parameters. The reach parameter may indicate an ability of the cutting blade to cut the bone surface using one or more dimensions, one or more surgical instrument limitation parameters, and one or more cutting blade parameters. The method may also include performing, in accordance with the reach parameter, alignment of the cutting blade to the bone surface.

In any of the preceding or subsequent examples, performing may include automatically performing, in accordance with the reach parameter, the alignment of the cutting blade.

In any of the preceding or subsequent examples, the alignment of the cutting blade to the bone surface may be performed using at least one of: the implant surface plane, the another implant surface plane, or any combination thereof.

In any of the preceding or subsequent examples, the alignment of the cutting blade to the bone surface may be performed using a tangent line of at least one of: the implant surface plane, the another implant surface plane, or any combination thereof, wherein the bone surface is at least one of: a splined bone surface, an analytically defined surface, or any combination thereof.

In any of the preceding or subsequent examples, the method may further include actuating, using at least one processor, the cutting blade in accordance with the reach parameter.

In any of the preceding or subsequent examples, the method may include removing at least a portion in a plurality of portions of the bone surface using the alignment of cutting blade.

In any preceding or subsequent examples, the reach parameter may indicate an inability of the cutting blade to cut the bone surface using the implant surface plane upon the orthogonal projection not intersecting any splined implant surfaces in the plurality of splined implant surfaces, wherein the actuating may include preventing cutting by the cutting blade.

In any preceding or subsequent examples, the reach parameter may indicate an ability of the cutting blade to cut the bone surface using at least one splined implant surface in the plurality of splined implant surfaces upon the orthogonal projection intersecting the at least one splined implant surface, wherein the actuating may include aligning the cutting blade using the splined implant surface, selecting a side of the cutting blade proximate to the splined implant surface, and actuating the cutting blade for cutting the bone surface using the selected side.

In any preceding or subsequent examples, the reach parameter may indicate an ability of the cutting blade to cut the bone surface using two or more splined implant surfaces in the plurality of splined implant surfaces upon the orthogonal projection intersecting two or more splined implant surfaces, wherein the actuating may include identifying a first splined implant surface in two or more splined implant surfaces based on at least one of: a smallest distance representative of a distance from the reference location on the cutting blade to at least one transition edge of the first splined implant surface, a smallest rotational angle of the cutting blade, or any combination thereof, aligning the cutting blade using the first splined implant surface, selecting a side of the cutting blade proximate to the first splined implant surface, and actuating the cutting blade for cutting the bone surface using the selected side.

In any preceding or subsequent examples, the surgical instrument is a surgical saw.

In any preceding or subsequent examples, one or more dimensions may include one or more plane coordinates associated with at least one transition edge between at least one splined implant surface and at least splined another implant surface in a plurality of splined implant surfaces of the implant.

In any preceding or subsequent examples, the reach parameter may be determined based on a combination of a minimum absolute distance between the distal end of the cutting blade and one or more coordinates associated with the transition edge and a negative normal distance, the negative normal distance is determined using a difference between the normal distance and the reference distance and the negative direction of cutting of the cutting blade.

In some examples, the current subject matter relates to a surgical system. The system may include a surgical instrument having a cutting blade for cutting a bone surface in a bone of a patient corresponding to an implant surface plane of an implant. The implant surface plane may be defined by at least one transition edge separating the implant surface plane from another implant surface plane of the implant, the implant is configured for implantation into the bone. The system may include at least one processor and at least one memory storing instructions that, when executed by the at least one processor, cause the at least one processor to: receive one or more dimensions associated with the implant surface plane, identify one or more surgical instrument limitation parameters and one or more cutting blade parameters including a reference distance representative of a distance from a reference location on the cutting blade to the at least one transition edge, a normal distance from the at least one transition edge to a surface of the cutting blade, and a direction of cutting of the cutting blade, determine a reach parameter of the cutting blade in relation to the bone surface based on one or more dimensions, one or more surgical instrument limitation parameters, and one or more cutting blade parameters, the reach parameter indicating an ability of the cutting blade to cut the bone surface using the one or more dimensions, one or more surgical instrument limitation parameters, and one or more cutting blade parameters, and perform, in accordance with the reach parameter, alignment of the cutting blade to the bone surface.

Examples of the present disclosure provide numerous advantages. For instance, the current subject matter may be configured to more precisely determine when a surgical cutting instrument's cutting blade is properly aligned with and/or is able to cut a particular bone surface plane corresponding to a surface plane of the implant. Such determination allows operators of the surgical instrument systems to avoid improper and/or unnecessary resections of tissue, thereby allowing for installation of thinner implants into bones of patients.

Further features and advantages of at least some of the examples of the current subject matter, as well as the structure and operation of various examples of the current subject matter, are described in detail below with reference to the accompanying drawings.

### BRIEF DESCRIPTION OF THE DRAWINGS

The accompanying drawings, which are incorporated in and constitute a part of this specification, show certain features of the subject matter disclosed herein and, together with the description, help explain some of the principles associated with the disclosed implementations. In the drawings,

FIG. 1 illustrates an example computer-assisted surgical system (CASS), according to some examples of the current subject matter;

FIG. 2 is a block diagram depicting an example system for performing a robotically assisted surgical procedure;

FIG. 3 illustrates an example surgical resection device, according to some examples of the current subject matter;

FIG. 4 illustrates further details of the surgical resection device being coupled to an optical tracking system, according to some examples of the current subject matter;

FIG. 5 illustrates further details of the surgical resection device having a handle and as part of a navigation system including an optical tracking system coupled and/or fixed to patient anatomy to be resected, according to some examples of the current subject matter;

FIG. 6 illustrates an example operation of the current subject matter, according to some examples;

FIG. 7 illustrates examples of normal distances between the cutting blade and one or more transition edges, according to some examples of the current subject matter;

FIG. 8A illustrates an example outcome of the determinations performed by the surgical computer of the CASS, according to some examples of the current subject matter;

FIG. 8B illustrates another example outcome of the determinations performed by the surgical computer of the CASS, according to some examples of the current subject matter;

FIG. 8C illustrates yet another example outcome of the determinations performed by the surgical computer of the CASS, according to some examples of the current subject matter;

FIG. 9A illustrates another example outcome of the determinations performed by the surgical computer, according to some examples of the current subject matter;

FIG. 9B illustrates another example outcome of the determinations performed by the surgical computer, according to some examples of the current subject matter;

FIG. 10A illustrates an example of an alignment determination performed by the surgical computer, according to some examples of the current subject matter;

FIG. 10B illustrates another example of an alignment determination performed by the surgical computer, according to some examples of the current subject matter;

FIG. 10C illustrates yet another example of an alignment determination performed by the surgical computer, according to some examples of the current subject matter;

FIG. 11 illustrates an example of a multi-stage bone cutting process, according to some embodiments of the current subject matter;

FIG. 12 illustrates an example process for positioning and operating a surgical instrument, according to some examples of the current subject matter;

FIG. 13 illustrates another example process for positioning and operating a surgical instrument, according to some examples of the current subject matter;

FIG. 14 illustrates an example of a storage medium to store logic in accordance with one or more features of the present disclosure; and

FIG. 15 illustrates an example computing platform in accordance with one or more features of the present disclosure.

It should be understood that the drawings are not necessarily to scale and that the disclosed examples are sometimes illustrated diagrammatically and/or in partial views. In certain instances, details that are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

To address these and potentially other deficiencies of currently available solutions, one or more implementations of the current subject matter relate to methods, systems, articles of manufacture, and the like that can, among other possible advantages, provide a system and a method for controlling operation of a surgical system.

In some examples, the current subject matter relates to a surgical system that may be used to perform various surgical procedures, including, for instance, but not limited to, a total knee arthroplasty (TKA), a hip replacement, a shoulder replacement, and/or any other procedures and/or any combination of procedures involving removal of bony tissue. In addition to various components that will be discussed below, the surgical system may include a robotic surgical instrument that may have an attachment, an accessory, a tool, etc., such as, for instance, a cutting blade for cutting a bone surface in a bone of a patient. The surgical instrument may use the cutting blade to make incisions and/or cuts that may correspond to one or more surface planes of an implant that is to be positioned in the bone of the patient. An implant surface plane may be defined by one or more transition edges that may separate the implant surface plane from another implant surface plane of the implant. In some examples, the current subject mater may be configured to perform a surgical cutting procedure, where it may be desirable to create a curved surface rather than a flat plane (for instance, where an “ultra-congruent” implant is used to allow the curved outline to closely match the outline of the resected bone). Such curved surface is referred to as a “ruled surface” and/or a “scroll”, which allows a straight line to be drawn at any point on the surface. Typical examples of ruled surfaces include cones, cylinders, and/or any other geometric shapes.

The surgical system may also include one or more processing components that may assist in determining how incisions and/or cuts in the bone of the patient need to be made. To do so, the processor may perform various analyses that may involve assessment of one or more parameters associated with the implant (e.g., coordinates and/or dimensions of the implant, implant surface planes, angles, etc.), the bone of the patient, one or more parameters associated with the cutting blade (e.g., faces of the blade, distances to/from the blade from/to implant surface planes, transition edges, motor speed, power, surgical instrument status, etc.), and/or any other data. The processor may also analyze various data (e.g., optical, audio, visual, graphical, image, etc.) that may be provided to the system (e.g., from a storage, via a network connection, etc.) and/or obtained by one or more sensors, which may be part of and/or separate from the surgical system. The data may relate patient and/or patient's extremities' position(s) prior to, during, and/or after surgical procedure performed using the surgical system.

In operation, the surgical system, and in particular, its processor may be configured to receive one or more dimensions associated with the implant surface plane of the implant. The dimensions may be representative of the size of the implant, coordinates of one or more implant surface plane(s), etc. For instance, the dimension(s) may include one or more plane coordinates associated with at least one transition edge between at least one implant surface plane and at least another implant surface plane in a plurality of implant surface planes of the implant. Further, the processor may receive data, information, etc. about the surgical system, and in particular, its surgical instrument that is used with the cutting blade to perform cutting, which may include various limitations (e.g., mechanical, electrical, electro-mechanical, dimensional, spatial, etc. limitations), movement and/or positional constraints (e.g., rotations, translations, etc. that might not be possible), etc. of the surgical instrument. These may be collectively referred to as surgical instrument limitation parameters. Moreover, the processor of the surgical system may be configured to identify one or more cutting blade parameters. These may include, but are not limited to, a reference distance representative of a distance from a reference location on the cutting blade to at least one transition edge separating implant surface planes, a normal distance from at least one transition edge to a surface of the cutting blade, a direction of cutting of the cutting blade, and/or any other parameters, and/or any combination thereof. The reference location on the cutting blade may be selected by the processor and/or by the user of the surgical system. The cutting blade parameters may be determined for one or more implant surface planes and/or for a single implant surface plane. The cutting blade parameters and/or the surgical instrument limitation parameters may be stored by the surgical system and retrieved by the processor upon generating an appropriate query to a storage location where such parameters are stored. Alternatively, or in addition, the parameters may be provided to the processor for analysis.

Once the dimensions of the implant surface plane(s), the surgical instrument limitation parameters, and cutting blade parameter(s) are provided to the processor, the processor may be configured to determine a reach parameter of the cutting blade in relation to the bone surface using the provided dimensions, the surgical instrument limitation parameters, and the cutting blade parameters. The reach parameter may indicate an ability of the surgical instrument to place the cutting blade on the planned cut surface of the bone in accordance with dimensions of the implant surface plane and one or more surgical instrument limitation parameter(s). Depending on the reach parameter, the processor may be configured to determine whether or not to actuate the cutting blade. Actuation of the cutting blade may include, for example, actuation of various operational components of the surgical system (e.g., motor components, surgical instrument, etc.) that may be used to position the cutting blade for cutting. Alternatively, or in addition, actuation of the cutting blade may refer to a determination (e.g., by the processor) whether the surgical instrument is active (e.g., operational, is in the ON state, etc.) and may perform cutting. As can be understood, the current subject is not limited to these options.

In some examples, the reach parameter may indicate an inability of the cutting blade to cut the bone surface using the implant surface plane. If so, the processor may be configured to prevent cutting by the cutting blade (e.g., prevent supplying electrical signals triggering operation of the cutting blade). Alternatively, if the reach parameter indicates an ability of the cutting blade to cut the bone surface using the implant surface plane. The processor may be configured to cause alignment of the cutting blade by the robotic actuator in relation to the implant surface plane. Once the cutting blade is aligned, the processor may be configured to select a specific side of the cutting blade for performing cutting. The selected side may be proximate to the bone surface that may correspond to the implant surface plane. Upon alignment of the cutting blade and selection of the specific side of the cutting blade, the processor may be configured to transmit one or more electrical signals to one or more electronic components (e.g., motors, etc.) responsible for operating the cutting blade to initiate operation of the cutting blade and perform requisite cuts of the bone of the patient that may correspond to the implant surface plane.

In some alternate, non-limiting, examples, the processor may determine a reach parameter that may indicate an ability of the cutting blade to cut the bone surface using a plurality of implant surface planes of the implant. In this case, the processor may be configured to identify a first implant surface plane in the plurality of implant surface planes based on at least one of: a smallest distance representative of a distance from the reference location on the cutting blade to at least one transition edge of the first implant surface plane, a smallest rotational angle of the cutting blade, and/or any combination thereof. The first implant surface plane may be the closest implant surface plane corresponding to the bone surface of the bone to the cutting blade. The processor may then cause alignment of the cutting blade using the first implant surface plane and selection of a side of the cutting blade proximate to the first implant surface plane. Once alignment and selection operations are completed, the processor may be configured actuate the cutting blade.

In some examples, the reach parameter may be determined based on a combination of a minimum absolute distance between the distal end of the cutting blade and one or more coordinates associated with the transition edge and a negative normal distance. The negative normal distance may be determined using a difference between the normal distance and the reference distance as well as the negative direction of cutting of the cutting blade (e.g., represented by a vector in relation to direction of the saw in relation to a specific transition edge and/or any other point(s)/location(s)/coordinate(s)).

In some examples, the surgical system may be configured to performing cutting of curved surfaces using the cutting blade. Each such surface may be separated into a plurality of splined implant surfaces and the above process may be used to analyze each splined implant surface. Alternatively, or in addition, a spline fitting algorithm may be used to determine how the cutting blade should be actuated with regard to each surface, which may use a spline equation that is precalculated (on a per implant basis) by sampling a predetermined number (e.g., N) of points on the curved surface. Further, in some non-limiting examples, an analytical function may be used to define a surface (e.g., geometric (e.g., circle), polygonal, etc.) for cutting by the cutting blade.

Similar to the process above, the processor of the surgical system may be configured to receive one or more dimensions associated with an implant surface plane defined by a plurality of splined implant surfaces. Each splined implant surface may include at least one transition edge separating one splined implant surface from another splined implant surface. The surgical system may use the cutting blade to cut a bone surface in the bone that may correspond to at least one splined implant surface. The processor may then identify one or more surgical instrument limitation parameters and/or one or more cutting blade parameters, as discussed above, e.g., a reference distance representative of a distance from a reference location on the cutting blade to at least one transition edge of the splined implant surface, a normal distance from at least one transition edge to a surface of the cutting blade, a direction of cutting of the cutting blade, and/or any combination thereof. The reference distance may be determined based on an orthogonal projection to the implant surface plane. The processor may then determine a reach parameter of the cutting blade in relation to the bone surface based on the dimensions, the surgical instrument limitation parameter(s) and the cutting blade parameter(s). The reach parameter may be indicative of whether or not to actuate (as discussed herein) the cutting blade in relation to one or more splined surfaces.

In some examples, the reach parameter may indicate an inability of the cutting blade to cut the bone surface using the implant surface plane upon the orthogonal projection not intersecting any splined implant surfaces. In this case, the processor may prevent operation of the cutting blade, and thus, no cutting by the cutting blade will be performed.

Alternatively, the reach parameter may indicate an ability of the cutting blade to cut the bone surface using at least one splined implant surface upon the orthogonal projection intersecting at least one splined implant surface. In this case, the processor may be configured to cause alignment of the cutting blade using the splined implant surface, selection of a side of the cutting blade proximate to the splined implant surface, and, subsequently, actuation of the cutting blade for cutting the bone surface using the selected side.

In one or more alternate, non-limiting examples, the reach parameter may indicate an ability of the cutting blade to cut the bone surface using two or more splined implant surfaces upon the orthogonal projection intersecting such two or more splined implant surfaces In this case, the processor may be configured to identify a first splined implant surface in the two or more splined implant surfaces based on at least one of: a smallest distance representative of a distance from the reference location on the cutting blade to at least one transition edge of the first splined implant surface, a smallest rotational angle of the cutting blade, and/or any combination thereof. Once identification of the first splined implant surface is made, the processor may cause alignment of the cutting blade using the first splined implant surface, selection of a side of the cutting blade proximate to the first splined implant surface, and actuation of the cutting blade for cutting the bone surface using the selected side.

In some examples, the surgical system may be configured to use the processes discussed herein to perform cutting of the bone in multiple stages. This may be helpful when various bone dimensions, surgical instrument limitation parameters, and/or any other constraints, etc. may limit operational capabilities of the cutting blade (e.g., rotation of the cutting blade to a predetermined degree preventing the cutting blade to reach a particular surface (e.g., a curved surface). Using the multi-stage approach, the current subject matter's surgical system may be configured to perform a predetermined number of planar bone cuts or “rough cuts” to remove tissue along one or more planes. How such rough cutting may be performed using determined by the one or more iterations of the processes described herein. Once rough cuts are completed, more finer cuts along a predetermined bone contour (e.g., as may be defined by one or more implant surfaces) may be performed. Similarly, how the finer cuts may be performed may be defined by one or more further iterations of the processes described herein. Such multi-stage cutting may reduce cutting blade's inability to reach certain surfaces and allow the cutting blade to more precisely cut the same.

In some examples, the current subject matter's surgical system may be configured to operate using a hand-held and/or surgeon-guided robotic instrument. Alternatively, or in addition, the processes described herein may be similarly applicable to automated robotic instruments.

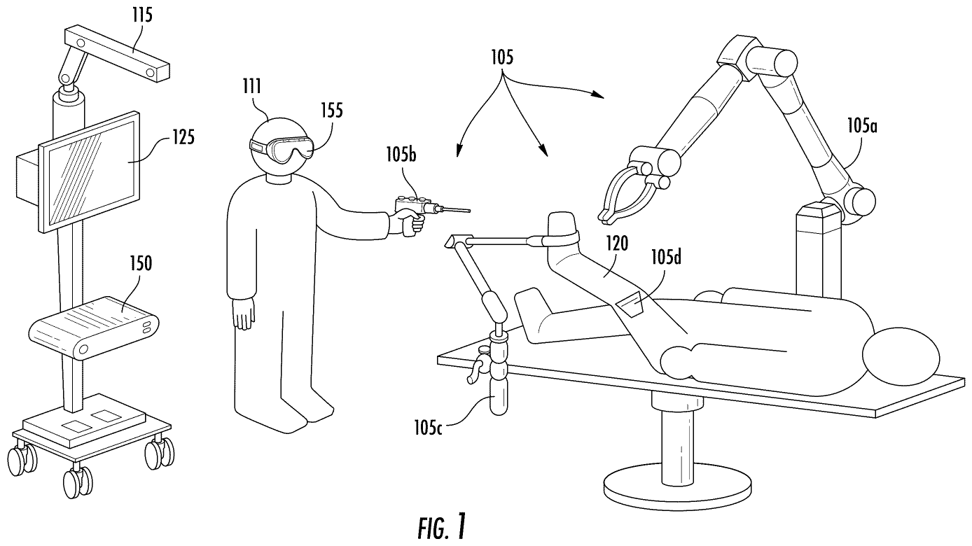

FIG. 1 illustrates an example computer-assisted surgical system (CASS) 100, according to some examples of the current subject matter. The CASS may use computers, robotics, and/or imaging technology to aid surgeons in performing orthopedic surgery procedures such as total knee arthroplasty (TKA) or total hip arthroplasty (THA). For example, surgical navigation systems may aid surgeons in locating patient anatomical structures, guiding surgical instruments, implanting medical devices with a high degree of accuracy, and/or any other procedures. Surgical navigation systems such as the CASS 100 may employ various forms of computing technology to perform a wide variety of standard and minimally invasive surgical procedures and techniques. Moreover, these systems may allow surgeons to more accurately plan, track and navigate the placement of instruments and implants relative to the body of a patient, as well as conduct pre-operative and intra-operative body imaging. The CASS 100 may include one or more of the following: an effector platform 105, a robotic arm 105a, an end effector 105b, a limb positioner 105c, a cutting guide or jig 105d, a tracking system 115, a display 125, a surgical computer 150, and a navigation system 120. A surgeon 111 (and/or any other medical professional) may perform a procedure using the CASS 100. In some cases, the surgeon 111 may wear one or more augmented reality head mounted displays, such as, for instance, an AR HMD 155.

The effector platform 105 may position surgical tools relative to a patient during a medical procedure, e.g., a surgery. The components of the effector platform 105 may vary, depending on implementations and/or uses of CASS 100. For example, for a knee surgery, the effector platform 105 may include the end effector 105b that may hold surgical tools and/or instruments during their use. The end effector 105b may be a handheld device or instrument used by the surgeon 111 (e.g., a CORI® hand piece or a cutting guide or jig) and/or, alternatively, or in addition, the end effector 105b may include a device or instrument held or positioned by the robotic arm 105a. While one robotic arm 105a is illustrated in FIG. 1, as may be understood, in some examples, there may be multiple robotic arms and/or devices. As examples, there may be one robotic arm 105a on each side of an operating table and/or two devices on one side of the table. The robotic arm 105a may be mounted directly to the table, be located next to the table on a floor platform (not shown), mounted on a floor-to-ceiling pole, and/or mounted on a wall or ceiling of an operating room, and/or coupled and/or positioned in any other way. The floor platform may be fixed and/or moveable. In some examples, the robotic arm 105a may be mounted on a floor-to-ceiling pole located between the patient's legs or feet. In some examples, the end effector 105b may include a suture holder or a stapler to assist in closing wounds. Further, in the case of two robotic arms 105a, the surgical computer 150 may operate the robotic arms 105a to work together to suture a wound at closure. Alternatively, or in addition, the surgical computer 150 may operate one or more robotic arm 105a to staple the wound at closure.

The effector platform 105 may include the limb positioner 105c for positioning the patient's limbs during surgery. The limb positioner 105c may be operated manually by the surgeon 111 and/or alternatively, or in addition, change limb positions based on instructions received from the surgical computer 150. While one limb positioner 105c is illustrated in FIG. 1, as may be understood, the CASS 100 may include multiple limb positioners 105c and/or devices. For instance, there may be one limb positioner 105c on each side of the operating table and/or two devices on one side of the table. The limb positioner 105c may be mounted directly to the table, be located next to the table on a floor platform (not shown in FIG. 1), mounted on a pole, and/or mounted on a wall or ceiling of an operating room, and/or coupled and/or positioned in any other way. In some examples, the limb positioner 105c may be used in non-conventional ways, such as, for instance, a retractor and/or specific bone holder. The limb positioner 105c may, for instance, include an ankle boot, a soft tissue clamp, a bone clamp, and/or a soft-tissue retractor spoon, such as a hooked, curved, or angled blade, and/or any other component. In some examples, the limb positioner 105c may include a suture holder to assist in closing wounds.

The effector platform 105 may include tools, such as a screwdriver, light and/or laser, to indicate an axis or plane, bubble level, pin driver, pin puller, plane checker, pointer, finger, or some combination thereof.

The CASS 100 may also include a resection equipment (not shown in FIG. 1) to perform bone and/or tissue resection using, for example, mechanical, ultrasonic, and/or laser techniques. Examples of resection equipment include drilling devices, burring devices, oscillatory sawing devices, vibratory impaction devices, reamers, ultrasonic bone cutting devices, radio frequency ablation devices, reciprocating devices (such as a rasp or broach), and/or laser ablation systems. In some examples, the resection equipment is held and operated by the surgeon during surgery. In other examples, the effector platform 105 may be used to hold the resection equipment during use.

The effector platform 105 may also include the cutting guide or jig 105D that may be used to guide saws and/or drills used to resect tissue during surgery. Such cutting guide or jig 105d may be formed integrally as part of the effector platform 105 and/or robotic arm 105a, and/or cutting guides may be separate structures that may be matingly and/or removably attached to the effector platform 105 and/or robotic arm 105a. The effector platform 105 and/or robotic arm 105a may be controlled by the CASS 100 to position a cutting guide or jig 105D adjacent to the patient's anatomy in accordance with a pre-operatively or intraoperatively developed surgical plan such that the cutting guide or jig 105d may produce a precise bone cut in accordance with the surgical plan.

The tracking system 115 may use one or more sensors to collect real-time position data that locates the patient's anatomy and surgical instruments. For example, for TKA procedures, the tracking system 115 may provide a location and orientation of the end effector 105b during the procedure. In addition to positional data, data from the tracking system 115 may also be used to infer velocity/acceleration of anatomy/instrumentation, which may be used for tool control. In some examples, the tracking system 115 may use a tracker array attached to the end effector 105b to determine the location and orientation of the end effector 105b. The position of the end effector 105b may be inferred based on the position and orientation of the tracking system 115 and a known relationship in three-dimensional space between the tracking system 115 and the end effector 105b. Various types of tracking systems may be used in various examples of the current subject matter including, but not limited to, infrared (IR) tracking systems, electromagnetic (EM) tracking systems, video or image based tracking systems, ultrasound registration and tracking systems, and/or any other types of tracking systems. Using the data provided by the tracking system 115, the surgical computer 150 may detect objects and/or prevent collision. For instance, the surgical computer 150 may prevent the robotic arm 105a and/or the end effector 105b from colliding with soft tissue.

Any suitable tracking system may be used for tracking surgical objects and patient anatomy in the surgical theatre. For instance, a combination of IR and visible light cameras may be used in an array. Various illumination sources, such as, an IR LED light source, may illuminate the scene allowing three-dimensional imaging to occur. In some examples, this may include stereoscopic, tri-scopic, quad-scopic, etc. imaging. In addition to the camera array, which in some examples, may be affixed to a cart, additional cameras may be placed throughout the surgical theatre. For instance, handheld tools and/or headsets worn by operators/surgeons may include imaging capability that communicates images back to a central processor to correlate those images with images captured by the camera array. This may give a more robust image of the environment for modeling using multiple perspectives. Further, some imaging devices may be of suitable resolution and/or have a suitable perspective on the scene to pick up information stored in quick response (QR) codes and/or barcodes. This may be helpful in identifying specific objects not manually registered with the system. In some examples, the camera may be mounted on the robotic arm 105a.

In some examples, specific objects may be manually registered by a surgeon with the system preoperatively and/or intraoperatively. For instance, by interacting with a user interface, a surgeon 111 may identify the starting location for a tool or a bone structure. By tracking fiducial marks associated with that tool and/or bone structure, and/or by using other conventional image tracking modalities, a processor may track that tool and/or bone as it moves through the environment in a three-dimensional model.

In some examples, certain markers, such as, fiducial marks that identify individuals, important tools, and/or bones in the theater may include passive and/or active identifiers that may be picked up by a camera and/or camera array associated with the tracking system. For example, an IR LED may flash a pattern that conveys a unique identifier to the source of that pattern, providing a dynamic identification mark. Similarly, one- or two-dimensional optical codes (barcode, QR code, etc.) may be affixed to objects in the theater to provide passive identification that may occur based on image analysis. If these codes may be placed asymmetrically on an object, they also may be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For instance, augmented reality headsets may be worn by surgeons and other staff to provide additional camera angles and tracking capabilities.

In addition to optical tracking, certain features of objects may be tracked by registering physical properties of the object and associating them with objects that may be tracked, such as, fiducial marks fixed to a tool and/or bone. For example, a surgeon may perform a manual registration process whereby a tracked tool and a tracked bone may be manipulated relative to one another. By impinging the tip of the tool against the surface of the bone, a three-dimensional surface may be mapped for that bone that is associated with a position and orientation relative to the frame of reference of that fiducial mark. By optically tracking the position and orientation (pose) of the fiducial mark associated with that bone, a model of that surface may be tracked with an environment through extrapolation.

The registration process that registers the CASS 100 to the relevant anatomy of the patient may also involve use of anatomical landmarks, such as, landmarks on a bone and/or cartilage. For example, the CASS 100 may include a 3D model of the relevant bone and/or joint and the surgeon 111 may intraoperatively collect data regarding the location of bony landmarks on the patient's actual bone using a probe that is connected to the CASS 100. Bony landmarks may include, for example, the medial malleolus and lateral malleolus, the ends of the proximal femur and distal tibia, and the center of the hip joint. The CASS 100 may compare and register the location data of bony landmarks collected by the surgeon with the probe with the location data of the same landmarks in the 3D model. Alternatively, or in addition, the CASS 100 may construct a 3D model of the bone and/or joint without pre-operative image data by using location data of bony landmarks and the bone surface that are collected by the surgeon using a CASS 100 probe and/or other means. The registration process may also include determining various axes of a joint. For example, for a TKA the surgeon 111 may use the CASS 100 to determine the anatomical and mechanical axes of the femur and tibia. The surgeon and the CASS 100 may identify the center of the hip joint by moving the patient's leg in a spiral direction (i.e., circumduction) so the CASS 100 may determine where the center of the hip joint is located.

A navigation system 120 may provide the surgeon with intraoperative, real-time visualization for the patient's bone, cartilage, muscle, nervous, and/or vascular tissues surrounding the surgical area. Examples of systems that may be employed for tissue navigation include fluorescent imaging systems and ultrasound systems.

The display 125 may provide graphical user interfaces (GUIs) that display images collected by the navigation system 120 as well other information relevant to the surgery. For example, the display 125 may overlay image information collected from various modalities (e.g., CT, MRI, X-ray, fluorescent, ultrasound, etc.) collected pre-operatively or intra-operatively to give the surgeon various views of the patient's anatomy as well as real-time conditions. The display 125 may include, for example, one or more computer monitors. Alternatively, or in addition, one or more members of the surgical staff may wear an Augmented Reality (AR) Head Mounted Device (HMD). For example, in FIG. 1 the surgeon 111 may wear AR HMD 155 that may, for example, overlay pre-operative image data on the patient or provide surgical planning suggestions. Various example uses of the AR HMD 155 in surgical procedures are detailed in the sections that follow.

The surgical computer 150 may provide control instructions to various components of the CASS 100, collects data from those components, and provides general processing for various data needed during surgery. In some examples, the surgical computer 150 may be a general-purpose computer. In other examples, the surgical computer 150 may be a parallel computing platform that uses multiple central processing units (CPUs) or graphics processing units (GPU) to perform processing. In some examples, the surgical computer 150 may be connected to a remote server over one or more computer networks (e.g., the Internet). The remote server may be used, for example, for storage of data or execution of computationally intensive processing tasks.

Various techniques generally known in the art may be used for connecting the surgical computer 150 to the other components of the CASS 100. Moreover, the computers may connect to the surgical computer 150 using a mix of technologies. For example, the end effector 105b may connect to the surgical computer 150 over a wired (i.e., serial) connection. The tracking system 115, navigation system 120, and/or display 125 may similarly be connected to the surgical computer 150 using wired connections. Alternatively, or in addition, the tracking system 115, navigation system 120, and/or display 125 may connect to the surgical computer 150 using wireless technologies such as, without limitation, Wi-Fi, Bluetooth, Near Field Communication (NFC), or ZigBee.

In some examples, the CASS 100 may include the robotic arm 105a that may serve as an interface to stabilize and/or hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The robotic arm 105a may have multiple degrees of freedom (like a Spider device), and/or have the ability to be locked in place (e.g., by a press of a button, voice activation, a surgeon removing a hand from the robotic arm, or other method).

In some examples, movement of the robotic arm 105a may be effectuated by use of a control panel built into the robotic arm system. For example, a display screen may include one or more input sources, such as physical buttons or a user interface having one or more icons, that direct movement of the robotic arm 105a. The surgeon or other healthcare professional may engage with the one or more input sources to position the robotic arm 105a when performing a surgical procedure.

A tool and/or an end effector 105b attached or integrated into the robotic arm 105a may include, without limitation, a burring device, a scalpel, a cutting device, a retractor, a joint tensioning device, or the like. In examples in which the end effector 105b is used, the end effector may be positioned at the end of the robotic arm 105a such that any motor control operations may be performed within the robotic arm system. In examples in which a tool is used, the tool may be secured at a distal end of the robotic arm 105a, but motor control operation may reside within the tool itself.

The robotic arm 105a may be motorized internally to both stabilize the robotic arm, thereby preventing it from falling and hitting the patient, surgical table, surgical staff, etc., and to allow the surgeon to move the robotic arm without having to fully support its weight. While the surgeon is moving the robotic arm 105a, the robotic arm may provide some resistance to prevent the robotic arm from moving too fast or having too many degrees of freedom active at once. The position and the lock status of the robotic arm 105a may be tracked, for example, by a controller or the surgical computer 150.

In some examples, the robotic arm 105a may be moved by hand (e.g., by the surgeon) or with internal motors into its ideal position and orientation for the task being performed. In some examples, the robotic arm 105a may be enabled to operate in a “free” mode that allows the surgeon to position the arm into a desired position without being restricted. While in the free mode, the position and orientation of the robotic arm 105a may still be tracked as described above. In some examples, certain degrees of freedom may be selectively released upon input from user (e.g., surgeon) during specified portions of the surgical plan tracked by the surgical computer 150. Designs in which a robotic arm 105a may be internally powered through hydraulics or motors or provides resistance to external manual motion through similar means may be described as powered robotic arms, while arms that are manually manipulated without power feedback, but which may be manually or automatically locked in place, may be described as passive robotic arms.

The robotic arm 105a and/or the end effector 105b may include a trigger or other means to control the power of a saw or drill. Engagement of the trigger or other means by the surgeon may cause the robotic arm 105a and/or end effector 105b to transition from a motorized alignment mode to a mode where the saw or drill is engaged and powered on. Additionally, the CASS 100 may include a foot pedal (not shown) that causes the system to perform certain functions when activated. For example, the surgeon may activate the foot pedal to instruct the CASS 100 to place the robotic arm 105a and/or end effector 105b in an automatic mode that brings the robotic arm and/or end effector into the proper position with respect to the patient's anatomy in order to perform the necessary resections. The CASS 100 may also place the robotic arm 105a and/or end effector 105b in a collaborative mode that allows the surgeon to manually manipulate and position the robotic arm and/or end effector into a particular location. The collaborative mode may be configured to allow the surgeon to move the robotic arm 105a and/or end effector 105b medially and/or laterally, while restricting movement in other directions. As discussed, the robotic arm 105a and/or end effector 105b may include a cutting device (saw, drill, and burr) and/or the cutting guide or jig 105d that will guide a cutting device. In some examples, movement of the robotic arm 105a and/or robotically controlled end effector 105b may be controlled entirely by the CASS 100 without any, or with only minimal, assistance or input from a surgeon or other medical professional. In still other examples, the movement of the robotic arm 105a and/or robotically controlled end effector 105b may be controlled remotely by a surgeon or other medical professional using a control mechanism separate from the robotic arm or robotically controlled end effector device, for example using a joystick or interactive monitor or display control device.

The examples below describe uses of the robotic device in the context of a hip surgery; however, it should be understood that the robotic arm may have other applications for surgical procedures involving knees, shoulders, etc.

The robotic arm 105a may be used for holding the retractor. For example, the robotic arm 105a may be moved into the desired position by the surgeon. At that point, the robotic arm 105a may lock into place. In some examples, the robotic arm 105a is provided with data regarding the patient's position, such that if the patient moves, the robotic arm can adjust the retractor position accordingly. In some examples, multiple robotic arms may be used, thereby allowing multiple retractors to be held or for more than one activity to be performed simultaneously (e.g., retractor holding & reaming).

The robotic arm 105a may also be used to help stabilize the surgeon's hand while making a femoral neck cut. In this application, control of the robotic arm 105a may impose certain restrictions to prevent soft tissue damage from occurring. For example, the surgical computer 150 may track the position of the robotic arm 105a as it operates. If the tracked location approaches an area where tissue damage is predicted, a command may be sent to the robotic arm 105a causing it to stop. Alternatively, or in addition, where the robotic arm 105a is automatically controlled by the surgical computer 150, the surgical computer 150 may ensure that the robotic arm is not provided with any instructions that cause it to enter areas where soft tissue damage is likely to occur. The surgical computer 150 may impose certain restrictions on the surgeon to prevent the surgeon from reaming too far into the medial wall of the acetabulum or reaming at an incorrect angle or orientation.

In some examples, the robotic arm 105a may be used to hold a cup impactor at a desired angle or orientation during cup impaction. When the final position has been achieved, the robotic arm 105a may prevent any further seating to prevent damage to the pelvis.

The surgeon may use the robotic arm 105a to position the broach handle at the desired position and allow the surgeon to impact the broach into the femoral canal at the desired orientation. In some examples, once the surgical computer 150 receives feedback that the broach is fully seated, the robotic arm 105a may restrict the handle to prevent further advancement of the broach.

The robotic arm 105a may also be used for resurfacing applications. For example, the robotic arm 105a may stabilize the surgeon while using traditional instrumentation and provide certain restrictions or limitations to allow for proper placement of implant components (e.g., guide wire placement, chamfer cutter, sleeve cutter, plan cutter, etc.). Where only a burr is employed, the robotic arm 105a may stabilize the surgeon's handpiece and may impose restrictions on the handpiece to prevent the surgeon from removing unintended bone in contravention of the surgical plan.

The robotic arm 105a may be a passive arm. Alternatively, or in addition, the robotic arm 105a may be an intelligent holding arm. As can be understood, any other types of robotic arms may be used.

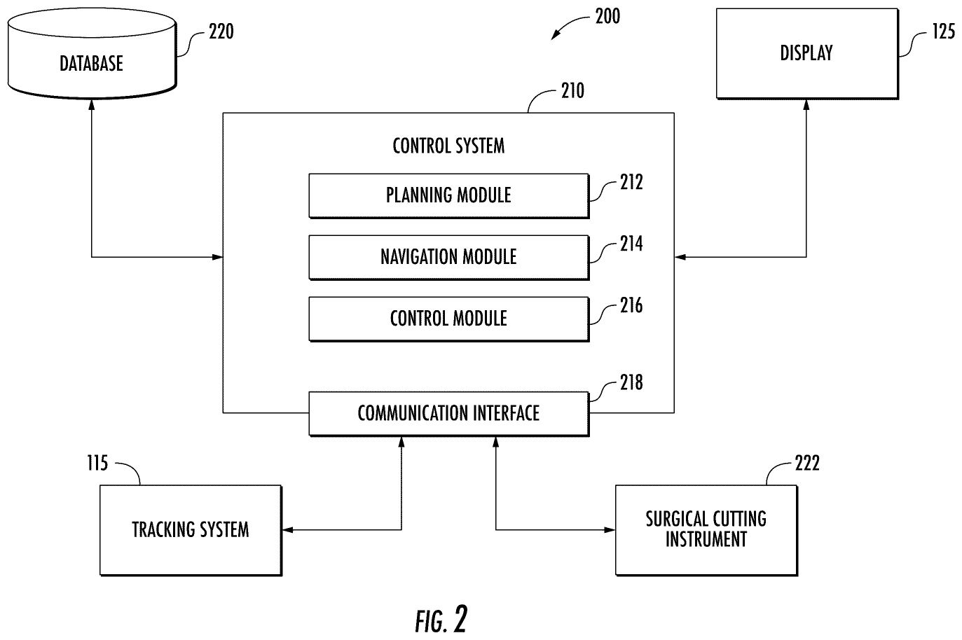

FIG. 2 is a block diagram depicting an example system 200 for performing a robotically assisted surgical procedure. The system 200 may be incorporated into the CASS 100 shown in FIG. 1 and used by the current subject matter system to control operation of one or more surgical components (e.g., surgical cutting instrument).

In some examples, the system 200 may include a control system 210, the tracking system 115, and the surgical cutting instrument 222 (e.g., end effector 105b). Optionally, the system 200 may include a display 125 and a database 220. In some examples, these components may be combined to provide navigation and control of the surgical cutting instrument 222, which may include navigation and control of a cutting tool 222 and/or a point probe, among other things, which may be used during an orthopedic surgery (and/or any other surgery).

One or more components of the system shown in FIG. 2 may be communicatively coupled using one or more communications networks. The communications networks may include one or more of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof.

Further, one or more components of the system shown in FIG. 2 may include any combination of hardware and/or software. In some examples, one or more components of the system may be disposed on one or more computing devices, such as, server(s), database(s), personal computer(s), laptop(s), cellular telephone(s), smartphone(s), tablet computer(s), virtual reality devices, and/or any other computing devices and/or any combination thereof. In some examples, one or more components of the system may be disposed on a single computing device and/or may be part of a single communications network. Alternatively, or in addition to, such devices may be separately located from one another. A device may be a computing processor, a memory, a software functionality, a routine, a procedure, a call, and/or any combination thereof that may be configured to execute a particular function associated with interface and/or document certification processes disclosed herein.

In some examples, one or more components of the system shown in FIG. 2 may include network-enabled computers. As referred to herein, a network-enabled computer may include, but is not limited to a computer device, or communications device including, e.g., a server, a network appliance, a personal computer, a workstation, a phone, a smartphone, a handheld PC, a personal digital assistant, a thin client, a fat client, an Internet browser, or other device. One or more components of the system also may be mobile computing devices, for example, an iPhone, iPod, iPad from Apple® and/or any other suitable device running Apple's iOS® operating system, any device running Microsoft's Windows®. Mobile operating system, any device running Google's Android® operating system, and/or any other suitable mobile computing device, such as a smartphone, a tablet, or like wearable mobile device.

One or more components of the system shown in FIG. 2 may include a processor and a memory, and it is understood that the processing circuitry may contain additional components, including processors, memories, error and parity/CRC checkers, data encoders, anti-collision algorithms, controllers, command decoders, security primitives and tamper-proofing hardware, as necessary to perform the interface and/or document certification functions described herein. One or more components of the system may further include one or more displays and/or one or more input devices. The displays may be any type of devices for presenting visual information such as a computer monitor, a flat panel display, and a mobile device screen, including liquid crystal displays, light-emitting diode displays, plasma panels, and cathode ray tube displays. The input devices may include any device for entering information into the user's device that is available and supported by the user's device, such as a touchscreen, keyboard, mouse, cursor-control device, touchscreen, microphone, digital camera, video recorder or camcorder. These devices may be used to enter information and interact with the software and other devices described herein.

In some examples, one or more components of the system shown in FIG. 2 may execute one or more applications, such as software applications, that enable, for instance, network communications with one or more components of system and transmit and/or receive data.

One or more components of the system shown in FIG. 2 may include and/or be in communication with one or more servers via one or more networks and may operate as a respective front-end to back-end pair with one or more servers. One or more components of the system may transmit, for example from a mobile device application (e.g., executing on one or more user devices, components, etc.), one or more requests to one or more servers. The requests may be associated with retrieving data from servers. The servers may receive the requests from the components of the system. Based on the requests, servers may be configured to retrieve the requested data from one or more storage locations. Based on receipt of the requested data from the databases, the servers may be configured to transmit the received data to one or more components of the system, where the received data may be responsive to one or more requests.

The system shown in FIG. 2 may include one or more networks. In some examples, networks may be one or more of a wireless network, a wired network or any combination of wireless network and wired network and may be configured to connect the components of the system and/or the components of the system to one or more servers. For example, the networks may include one or more of a fiber optics network, a passive optical network, a cable network, an Internet network, a satellite network, a wireless local area network (LAN), a metropolitan area network (MAN), a wide area network (WAN), a virtual local area network (VLAN), an extranet, an intranet, a Global System for Mobile Communication, a Personal Communication Service, a Personal Area Network, Wireless Application Protocol, Multimedia Messaging Service, Enhanced Messaging Service, Short Message Service, Time Division Multiplexing based systems, Code Division Multiple Access based systems, D-AMPS, Wi-Fi, Fixed Wireless Data, IEEE 802.11b, 802.15.1, 802.11n and 802.11g, Bluetooth, NFC, Radio Frequency Identification (RFID), Wi-Fi, and/or any other type of network and/or any combination thereof.

In addition, the networks may include, without limitation, telephone lines, fiber optics, IEEE Ethernet 802.3, a wide area network, a wireless personal area network, a LAN, or a global network such as the Internet. Further, the networks may support an Internet network, a wireless communication network, a cellular network, or the like, or any combination thereof. The networks may further include one network, or any number of the exemplary types of networks mentioned above, operating as a stand-alone network or in cooperation with each other. The networks may utilize one or more protocols of one or more network elements to which they are communicatively coupled. The networks may translate to or from other protocols to one or more protocols of network devices. The networks may include a plurality of interconnected networks, such as, for example, the Internet, a service provider's network, a cable television network, corporate networks, such as credit card association networks, and home networks.

The system shown in FIG. 2 may include one or more servers, which may include one or more processors that may be coupled to memory. Servers may be configured as a central system, server or platform to control and call various data at different times to execute a plurality of workflow actions. Servers may be configured to connect to the one or more databases. Servers may be incorporated into and/or communicatively coupled to at least one of the components of the system.

Further, one or more components of the system shown in FIG. 2 may be configured to execute one or more actions using one or more containers. In some examples, each action may be executed using its own container. A container may refer to a standard unit of software that may be configured to include the code that may be needed to execute the action along with all its dependencies. This may allow execution of actions to run quickly and reliably.

The control system 210 may include one or more computing devices configured to coordinate information received from the tracking system 115 and provide control to the surgical cutting instrument 222. In some examples, the control system 210 may include a planning module 212, a navigation module 214, a control module 216, and a communication interface 218. The planning module 212 can provide pre-operative planning capabilities that allow surgeons to virtually plan a procedure prior to reshaping a target joint during the surgical procedure on the patient.

In some examples, the planning module 212 may be used to manipulate a virtual model of the implant in reference to a virtual implant host model (such as, for instance, for the purposes of the TKA). The virtual model of the implant host (illustrating the joint to be replaced) may be created through use of a point probe or similar instrument tracked by the tracking system 115. The planning module 212 can collect data from surfaces of the target joint to recreate a virtual model of the patient's actual anatomical structure. By way of a non-limiting example, in a joint replacement surgery, this can increase accuracy of the planning process by using data collected after the joint has been exposed and without intra-operative imaging. Collecting surface data from the target bone(s) also can allow for iterative reshaping of the target bone to ensure proper fit of the prosthetic implants and optimization of anatomical alignment.

In some examples, the navigation module 214 can coordinate tracking the location and orientation of the implant, the implant host, and the surgical cutting instrument 222 during the surgical procedure. Further, the navigation module 214 can also coordinate tracking of the virtual models used during pre-operative or intra-operative planning within the planning module 212. Tracking the virtual models may include operations such as alignment of the virtual models with the implant host through data obtained via the tracking system 115. The navigation module 214 can receive input from the tracking system 115 regarding the physical location and orientation of the surgical cutting instrument 222 and an implant host. Tracking of the implant host may include tracking multiple individual bone structures, such as with patient tracking frames. For example, during a total knee replacement procedure, the tracking system 115 can individually track the femur and the tibia using tracking devices anchored to the individual bones (as shown, for example, in FIG. 1).

In some examples, the control module 216 can process information provided by the navigation module 214 to generate control signals for controlling the surgical cutting instrument 222. The control module 216 also can work with the navigation module 214 to produce visual animations to assist the surgeon during an operative procedure. Visual animations may be displayed via a display device, such as, for instance, display 125. In some examples, the visual animations may include real-time 3D representations of the implant, the implant host, and the surgical cutting instrument 222, among other things. Further, the visual animations may be color-coded to further assist the surgeon with positioning and orienting the implant.

The communication interface 218 can facilitate communication between the control system 210 and one or more external systems and/or devices. The communication interface 218 may include wired and/or wireless communication interfaces, such as Ethernet, IEEE 802.11 wireless, or Bluetooth, among others. As illustrated in FIG. 1, the primary external systems connected via the communication interface 218 may include the tracking system 115 and the surgical cutting instrument 222. Although not shown, the database 220 and the display 125, among other devices, also may be connected to the control system 210 via the communication interface 218. In some examples, the communication interface 218 can communicate over an internal bus to other modules and hardware systems within the control system 210.

The tracking system 115 can provide location and orientation information for surgical devices and parts of an implant host's anatomy to assist in navigation and control of semi-active robotic surgical devices. The tracking system 115 may include a tracker (e.g., patient tracking frames) that may include and/or otherwise provide tracking data based on one or more (e.g., three) positions and/or one or more (e.g., three) angles. The tracker may include one or more first tracking markers associated with the implant host and one or more second markers associated with the surgical device (e.g., surgical cutting instrument 222). The markers and/or some of the markers may be one or more of infrared sources, light emitting sources, radio frequency (RF) sources, ultrasound sources, and/or transmitters. The tracking system 115 may be an infrared tracking system, an optical tracking system, an ultrasound tracking system, an inertial tracking system, a wired system, an RF tracking system, and/or any other type of system and/or any combination thereof.

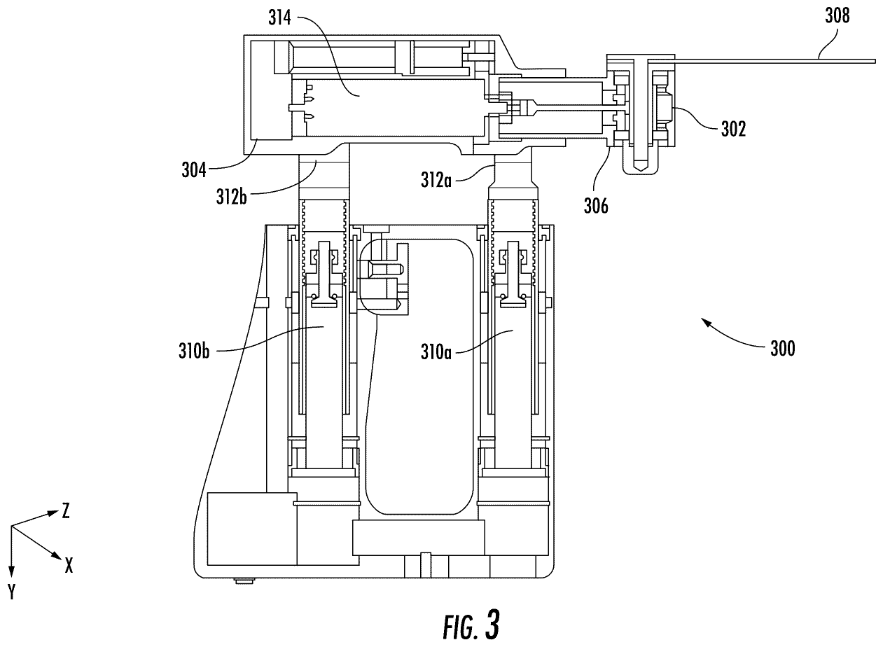

FIG. 3 illustrates an example surgical resection device 300, according to some examples of the current subject matter. The surgical resection device 300 may be handheld, may have a minimum of three degrees of freedom, and may be controlled through a navigation device in the CASS 100 (as shown in FIG. 1) to keep a cutting axis and/or plane constrained to a plane of interest, which may be determined preoperatively (referred to herein as a resection plane). The surgical resection device 300 may be determined to be aligned with the resection plane based on a cutting element of a resection tool 302 of the surgical resection device that is coupled to a housing 304 optionally via a flexible gasket 306. As shown in FIG. 3, the resection tool 302 may be an oscillating surgical saw that has a saw blade and/or cutting blade 308 cutting element. As can be understood, the resection tool may be another type of surgical saw (e.g., a sagittal surgical saw) and/or a burr, for example, and other types of cutting tools may also be used.

To constrain the resection tool 302 with respect to the resection plane, at least two linear actuators 310a-b may be coupled to the static housing 304. The linear actuators 310a-b may be capable of changing the position of the resection tool 302 in two degrees of freedom (i.e., a translational degree of freedom and a rotational degree of freedom). Accordingly, the linear actuators 310a-b may restrain four degrees of freedom while actively controlling two degrees of freedom. The linear actuators 310a-b may be coupled to the static housing 304 in this example via respective pinned linkages 312a-b and may be spaced from each other in a direction of a first axis (i.e., the Z axis illustrated in FIG. 3). The linear actuators 310a-b may be independently drivable to translate the resection tool 302 within a second axis (i.e., the Y axis illustrated in FIG. 3) and rotate the resection tool about a third axis (i.e., the X axis illustrated in FIG. 3).

In some examples, one or more of the linear actuators 310a-b may include a motor driving a nut that translates the rotational motion of the motor into a linear motion. In some examples, the nut may be supported by a sleeve. In some examples, the nut is configured to at least partial envelop the motor in at least some states of the linear actuator.

In some examples, the linear actuators 310a-b may be driven independently to allow for translation in the direction of the Y axis as well as adjustment in pitch via rotation about the X axis. The pinned linkages 312a-b therefore allow translation and rotation freedom in the Y direction and about the X axis, respectively. In some examples, the pinned linkages 312a-b may be static. In an example, one or more of the pinned linkages 312a-b may be configured to provide rotation while the other pinned linkage 312a-b may be configured to provide rotation and translations (e.g., a slot and pin). One or more of the linear actuators 310a-b may be a direct current stepper motor coupled to a nut and/or a lead screw assembly, a pneumatic actuator, a hydraulic actuator, a piezo-electric actuator, a rack and pinion actuator, or an actuator based on a crank/arm or cam/follower mechanism, for example, although other types of actuators can be used in other examples.

In some examples, including as illustrated in FIG. 3, the resection tool 302 may further be controlled in a third degree of freedom via a third rotational actuator, which is adjacent to a motor 314 (e.g., a direct current stepper motor) coupled to the static housing 304 in this example, although another type of rotational actuator can also be used. The third degree of freedom therefore in this example may be rotational and about the first axis (i.e., the Z axis illustrated in FIG. 3). In examples in which the cutting is performed by a burr, for example, the burr may inherently be rotating about the first axis (i.e., the Z axis in FIG. 3) to affect the resection of the patient anatomy and therefore a third rotational degree of freedom about the first axis for the resection tool 302 itself is not provided.

Accordingly, control in a third degree of freedom can be provided in examples in which the resection tool 302 may include the cutting blade 308, although the third degree of freedom may be controlled for other types of resection tools in other examples. In the example of the resection tool 302 with cutting blade 308 illustrated in FIG. 3, the motor 314 may facilitate rotation of the cutting blade 308 about the long axis of the cutting blade 308 (referred to herein as the first axis and is the Z axis, as shown in FIG. 3). Thus, the linear actuators 310a-b and motor 314 may be collectively configured to control the resection tool 302 in three degrees of freedom.

To facilitate the rotation of the resection tool 302 about the first axis (i.e., the Z axis in FIG. 3), the resection tool 302, in some examples, may include an annular gear (not shown in FIG. 3) and the resection tool 302 may be coupled to the static housing 304 via the flexible gasket 306 that allows relative motion between the static housing and the resection tool. In this example, the motor 314 may include a pinion gear (not shown in FIG. 3) that is configured to interface, through or within the static housing 304, with the annular gear of the resection tool 302 to rotate the resection tool about the first axis (i.e., the Z axis in FIG. 3). In other words, actuation of the motor 314, and thus the pinion gear, drives rotation of the resection tool 302 and associated cutting blade 308 within the static housing 304. Accordingly, in this example, the saw resection tool 302 (i.e., the oscillating surgical saw) is free to rotate in roll (i.e., about the first axis or the Z axis illustrated in FIG. 3) relative to the static housing 304.

In some examples (e.g., in which the resection tool includes a burr), the third degree of freedom may be translational and in the direction of the first axis (i.e., the Z axis illustrated in FIG. 3). In these examples, the motor 314 and/or another linear actuator is configured to extend and retract the resection tool 302 along a direction of the first axis.

Optionally, the resection tool 302 may be removable from the static housing 304. In these examples, particular degree(s) of freedom may be activated and/or inactivated based on the type or other characteristics of the installed resection tool 302. The activation or inactivation may be mechanical based on the connection or interface of the resection tool 302 (e.g., with respect to engagement with the motor 314 with the resection tool). In another example, the activation or inactivation can be electrical and managed by the surgical computer 150 and/or another device within the CASS 100 based on a determination of the type or other characteristics of the installed resection tool 302. Other methods for activating or inactivating degree(s) of freedom for particular installed resection tools may also be used in other examples.

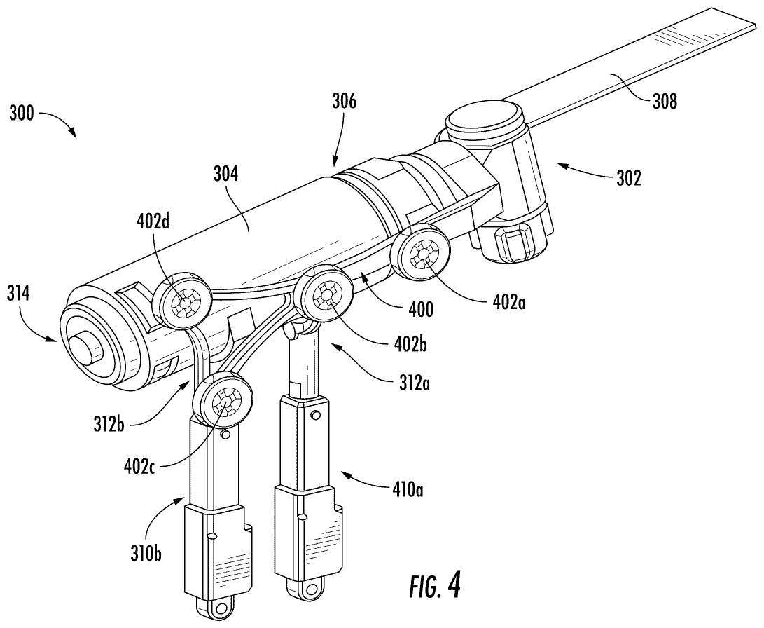

FIG. 4 illustrates further details of the surgical resection device 300 being coupled to an optical tracking system 400, according to some examples of the current subject matter. The optical tracking system 400 may include markers and/or fiducials 402a-d and may be coupled to at least the static housing 304 of the surgical resection device 300. As shown in FIG. 4, the position of the resection tool 302 may be determined based on motor encoders or other positions sensors. In other examples, the markers 402a-d may be coupled to mobile components (e.g., associated with the resection tool 302) with the actuation of the motor 314 and/or linear actuators 310a-b being based on the tracked position of the resection tool 302.

Other types of tracking devices may be used in other examples to track the position and orientation in space (e.g., the surgical environment) of the resection tool 302 by the CASS 100 and/or associated surgical computer 150 to facilitate control of the surgical resection device 300 with respect to its position, orientation, activation, and/or speed.

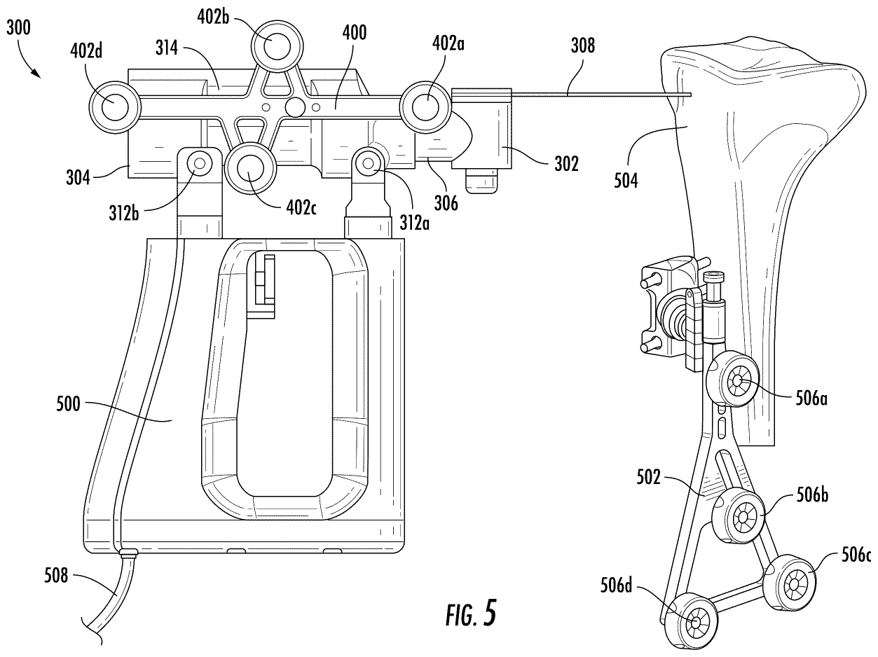

FIG. 5 illustrates further details of the surgical resection device 300 having a handle 500 and as part of a navigation system including an optical tracking system 502 coupled and/or fixed to patient anatomy 504 to be resected, according to some examples of the current subject matter. The handle 500 may be coupled to the static housing 304 and may provide a housing for the linear actuators 310a-b and/or pinned linkages 312a-b while providing a form factor that facilitates handheld operation of the surgical resection device 300. In some examples, at least one of the linkages 312a-b may be slotted to allow less constrained movement of the tool. Alternatively, or in addition, one or more of the linear actuators 310a-b may not be pinned, but, instead, may move only in one axis. In some examples, the housing may include a trigger. In some examples, the housing may include a gap between the linear actuators 310a-b. In further examples, the gap may be configured to fit the operator's fingers.

The optical tracking system 502 may include a plurality of markers or fiducials 506a-d that may allow the surgical computer 150 and/or other navigation and/or tracking device within the CASS 100 to determine the position and orientation of the patient anatomy 504. Accordingly, the optical tracking system 400 and/or 502 may facilitate relative determination of position and/or orientation within an operating environment to align the resection tool 302 of the surgical resection device 300 with a resection plane associated with the patient anatomy 504.

As shown in FIG. 5, the optical tracking system 400 may be coupled to the static housing 304. In some examples, the optical tracking system 400 may be coupled to the handle 500. In further examples, a tracking system may be coupled on multiple locations on the surgical resection device 300, such as, for instance, on both sides of the static housing 304 and/or the handle 500. Multiple tracking devices and/or systems may allow the surgical resection device 300 to continue to operate (i.e., maintain visibility of at least one tracking device by the tracking system 115) when the surgeon changes position and/or inverts the surgical resection device 300.

In some examples, the surgical resection device 300 may include a physical guide (not shown) statically affixed to the handle. The physical guide can reflect the natural plane and/or position of the cutting edge given no correction from the linear actuators 310 a-b and/or motor 314. The surgical resection device 300 may include one or more indices depicting the current correction provided by the linear actuators 310a-b and/or motor 314 in comparison to the physical guide. In some examples, the indices depict a maximum corrective capability.

The surgical resection device 300 may be coupled to the surgical computer 150 and/or another device with the CASS 100 via an electrical connection 508. In particular, the electrical connection 508 may be coupled to the linear actuators 310a-b and/or motor 314 to facilitating control or driving of those components by the surgical computer 150.

Existing robotic handheld systems, forming part of larger surgical platforms and/or distinct navigation systems may be used to perform orthopedic surgeries (e.g., TKA, shoulder replacement, etc.) and, typically, include robotic saw tools equipped with sawblades or cutting blades having three positioning degrees of freedom. This allows such tools to cut bone tissue so that an implant may be positioned in the bone of a patient. As may be understood, a sawblade/cutting blade can typically cut on a plane, and, as such, three degrees of freedom of movement is usually sufficient to robotically constrain the sawblade to a particular plane in space.

Regardless of the system within which such robotic tools are used, the saws can require that the system have six degrees of freedom navigational capabilities, a surgical plan, and a servo/motor system. A navigation system can provide real-time position of the robotic saw relative to a specific position of a certain anatomy of the patient. The surgical plan can provide a target surface to be resected on the anatomy, and a servo/motor system can actively control the tool's position to coincide with the surface defined in the surgical plan. Intrinsically, the robotic saw tool can only servo to and cut a plane. However, in certain scenarios, it may be desirable to create a curved surface rather than a flat plane. Such may be the case in which an “ultra congruent” implant is used which the resected bone closely matches the curved outline of the implant when viewed from the medial-lateral direction. As discussed herein, this type of surface can be denoted as a “ruled surface” and/or “scroll” since a straight line may be drawn at any point on the surface. Cones and cylinders are common examples of ruled surfaces.

In some examples, the current subject matter may be configured to perform cutting of ruled surfaces using a robotically controlled saw. Such robotically controlled saw cutting has numerous benefits over the existing systems. In particular, it allows removal of less bone and use of thinner implants so that more bone may be available for future revisions. Moreover, the current subject matter may provide reductions in stress shielding. Existing systems typically require 4-5 cuts for a TKA femoral component, which allows implants to be roughly 15-25 millimeters (mm) thick. However, as the thickness of the implant decreases, the number of cuts that is required for proper positioning of the implant increases.

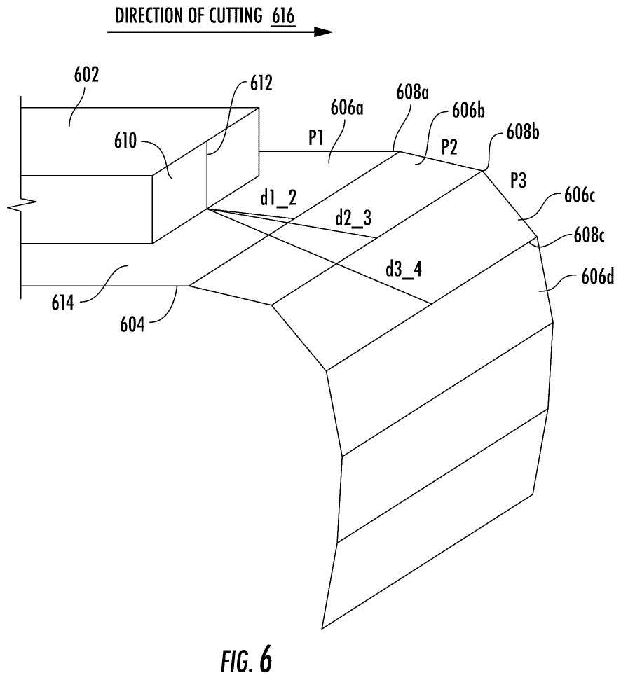

In some examples, prior to determining how surface cutting of a complex surface is going to be performed, the current subject matter may discretize a complex target surface using resolution n (e.g., by dividing the target surface into smaller planes, as for example, shown in FIG. 6). The result of discretizing is a set of small planes that may be cut with a using a robotic saw that has a limited number of degrees of freedom (e.g., 3). The current subject matter system may then determine (e.g., automatically) a correct plane to target for cutting. The system may, in addition to accounting for surgical instrument limitation parameters (e.g., defining various limitations of the surgical instrument), use a relationship of the cutting blade to the transition edges of intersecting planes to determine which plane to target for cutting. To do so, the current subject matter system may determine a distance from a reference point on the distal end of the cutting blade to a closest point on each transition edge and use it to determine proximity of the cutting blade to the transition edge. Then, the current subject matter system may determine normal distance(s) from the front face of the cutting blade to the transition edge(s). A positive normal distance may indicate that the transition edge has not yet been surpassed by the cutting blade. A negative normal distance may indicate that the transition edge has been passed by the cutting blade. The system may then use a combination of the minimum absolute distance and a negative normal distance (as defined by the direction of movement of the cutting blade) to identify the correct plane to target for cutting by the cutting blade.

Referring to FIG. 6, the operation of the current subject matter system in determining when a cutting blade (e.g., cutting blade 308 shown in FIG. 3) of a surgical instrument (e.g., resection tool 302 shown in FIG. 3) of surgical system (e.g., a CASS 100 shown in FIG. 1) may be actuated to cut a complex surface of a bone of a patient to position an implant. This determination may be performed by the surgical computer 150 integrated into the CASS 100 shown in FIG. 1. The surgical instrument includes a cutting blade 602 (similar to the cutting blade 308) that may be used to cut a bone surface in the bone corresponding to the implant surface 614.

The current subject matter may receive one or more dimensions associated with the implant surface 614 of the implant 604. The implant surface 614 may include one or more implant surface planes P1, P2, P3, . . . 406 (a, b, c, . . . ). Each implant surface plane may be defined by one or more transition edges 608 (a, b, c, d . . . ) that may separate one implant surface plane from another implant surface plane of the implant 604. For example, transition edge 608a may separate implant surface plane P1 606a and implant surface plane P2 606b; transition edge 608b may separate implant surface plane P2 606b and implant surface plane P3 606c; transition edge 608c may separate implant surface plane P3 606c and implant surface plane P4 606d; etc. The dimensions of each implant surface plane 606 and/or transition edges 608 may be provided to the surgical computer 150 for further processing. The dimensions may include coordinates, distances, lengths, areas, etc.

In some examples, the dimensions associated with the implant surfaces planes 606 and/or the transition edges 4608 may be provided to the CASS 100 as a set of input data. Alternatively, or in addition, the CASS 100 may be configured to ascertain each implant surface plane and/or each transition edge using one or more measurements, such as, for example, optical measurements performed using one or more optical sensors, mechanical measurements, and/or any other type of measurements.

In some examples, the surgical computer 150 may be configured to also determine and/or identify one or more surgical instrument limitation parameters associated with the surgical instrument (e.g., surgical resection device 300). These may include various mechanical, electrical, electro-mechanical, and/or any other operational limits of the surgical instrument that may prevent it from perform one or more operations (e.g., rotations, translations, and/or any other movements).

The surgical computer 150 may further identify one or more parameters of the cutting blade 602. The parameters may include a reference distance that may be representative of a distance (e.g., d1_2) from a reference location or reference point 612 on the cutting blade 602 to the at least one transition edge, e.g., transition edge 608a. The reference distances may be identified and/or determined to other transition edges. For example, distance d2_3 may be determined from the reference point 612 to the transition edge 608b; distance d3_4 may be determined from the reference point 612 to the transition edge 608c; etc. The surgical computer 150 may be configured to select a specific reference point 612 on the cutting blade 602 and determine reference distances from it accordingly. Alternatively, or in addition, the reference point 612 may be predetermined, e.g., a middle point on the cutting blade 602. As may be understood, any points on the cutting blade 602 may be selected as reference point 612.

Additional parameters of the cutting blade 602 may include a normal distance (e.g., Euclidean distance), which may be determined from at least one transition edge to a surface of or cutting blade face 610 of the cutting blade 602. The surgical computer 150 may be configured to determine normal distances to each transition edge 608 from the cutting blade face 610.

Further, the surgical computer 150 may be configured to determine another parameter of the cutting blade 602 corresponding to a direction (e.g., direction of cutting 616) of cutting by the cutting blade 602. This parameter may have a scalar value and a vector value.

Once the various dimensions associated with the implant 604, surgical instrument limitation parameters, and parameters of the cutting blade 602 are determined and/or identified, the surgical computer 150 may be configured to determine a reach parameter of the cutting blade 602 in relation to the bone surface of the bone where the implant 604 will be positioned. The reach parameter may be determined using one or more dimensions, surgical instrument limitation parameters, and/or one or more cutting blade parameters. The reach parameter may indicate an ability of the cutting blade 602 to cut the bone surface (e.g., reach) using the determined/identified dimension(s), surgical instrument limitation parameters, and/or cutting blade parameter(s). Depending on the reach parameter, the surgical computer 150 may be configured to determine whether or not to actuate the cutting blade 602. In some non-limiting examples, the reach parameter may be determined using a kinematic model of the surgical instrument. For instance, the kinematic model of a 3 degree of freedom surgical instrument may be used. As can be understood, any other types of models may be used.

In some examples, the reach parameter may be determined using dual quaternion technique. A dual quaternion may be defined as DQ=α+ϵα′, where α is the primary part and α′ is the dual part. The primary and dual parts are extracted with the P( ) and D( ) operators as follows: DQ=P(DQ)+ϵD(DQ). Primary and dual parts are quaternions with the form q=α+bî+cĵ+d\hat{k}, thus a complete dual quaternion is: h=α+bî+cĵ+d\hat{k}+ϵ(e+fî+gĵ+h\hat{k}).

The conjugate is defined as h*=Re(h)−Im(h), where Re and Im denote real and imaginary parts of the dual quaternion, respectively. Further, the norm of a quaternion is defined as ∥h∥=\sqrt{hh*}=\sqrt{h*h}. Also, the adjoint transformation is defined as Ad(r)p=rpr*, where r and p may represent dual quaternions. Finally, the sharp notation is defined as x^{#}=P(x)−εD(x) (a difference of the primary and dual components as opposed to a summation as noted above) and sharp adjoint transform defined as Ad#(x)y=x^{#}yx*.

A plane may be defined using dual quaternions as a unit vector normal to the plane and the perpendicular distance from the origin of a given coordinate system. Let n represent the plane unit normal, distance d=p·n where p is an arbitrary point on the plane. Thus, a plane P=n+εd=n+εq·n.

Using the above, kinematic equations may be expounded for the purposes of determining a reach parameter of the cutting blade by the surgical computer 150, as illustrated by the following example (here, an uppercase “T”′ may represent an affine transformation (as represented by a dual quaternion)). As an initial operation, the surgical computer 150 may derive transform the bone T_bone and saw body T_sawBody using data obtained from the tracking system and reported in the tracking reference frame and/or coordinate system. In this example, all coordinate transforms may be assumed to occur in the tracking system space.

The surgical computer 150 may then define a position of the cut plane as a midpoint of the target plane along with the plane normal. Additionally, the surgical computer 150 may define a plane as a point coincident with the target spline defined above with the normal being defined as a line perpendicular to the tangent line at that point. These representations may occur naturally in the implant coordinate space. Thus, the surgical computer 150 may transform the target cut plane to tracking space using the following:

$$P\_targetTrackingCS = Ad \# ( T\_boneTimplant ) P\_implant \qquad ( 1 )$$

Subsequently, the surgical computer 150 may transform the target plane to the saw coordinate system using the following:

$$P\_targetSawCS = Ad \# ( T\_saw ) P\_targetTrackingCS \qquad ( 2 )$$

Then, the surgical computer 150 may determine a current handle rotation relative to the surgical saw's body using the following:

$$θ = {sin}^{- 1} \frac{motor1 - motor2}{motorSeparation} . \qquad ( 3 )$$

Then, the surgical computer 150 may generate a dual quaternion for the handle transform using the following:

$$T\_sawToHandleRot = DQ ( cos ( θ ) , 0 , sin ( θ ) , 0 ) \qquad ( 4 )$$

It may also transform the target plane to the handle CS using:

$$P\_targetHandleCS = Ad \# ( T\_sawToHandleRot ) P\_targetSawCS \qquad ( 5 )$$

The surgical computer 150 may then determine intersection, Int1, of linear motor axes with the target plane for a first linear motor using:

$$Int 1 = P\_targetHandleCS [ 4 ] / P\_targetHandleCS [ 1 ] \qquad ( 6 )$$

where the surgical computer 150 may repeat the above for intersection, Int2, of linear motor axes with the target plane for a second linear motor.

The surgical computer 150 may then determine if intersections Int1 and Int2 are within mechanical limits of the linear motors.

It may also determine if the target plane is within mechanical limits of the rotational motor. To do so, the surgical computer may determine a dot product of the target plane normal with the neutral blade vector in the saw body coordinate system.

Lastly, the surgical computer 150 may determine the reach parameter as true if the target position is reachable by all motor axes.

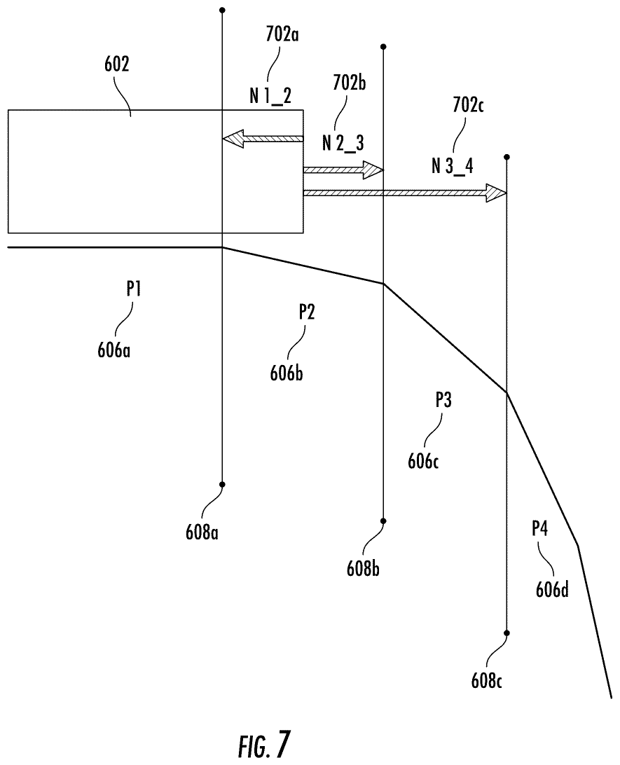

In some examples, the surgical computer 150 may determine the reach parameter based on a combination of a minimum absolute distance between the distal end of the cutting blade 602 and one or more coordinates associated with the transition edge (e.g., transition edge 608a) and a negative normal distance. The coordinates may define the location of the transition edge between one implant surface plane (e.g., implant surface plane P1 606a) and another implant surface plane (e.g., implant surface plane P2 606b) of the implant. The negative normal distance may be determined using a difference between the normal distance and the reference distance and the negative direction of cutting of the cutting blade, where the latter may be defined by a vector representative of a direction of movement of the cutting blade 602 (as shown in FIG. 7).

In some examples, the determined reach parameter may indicate an inability of the cutting blade 602 to cut the bone surface using the implant surface plane. In this scenario, the surgical computer 150 may be configured to prevent actuation (e.g., actuation of motors, positioning of the cutting blade, ability of the cutting blade to cut, etc.), and thus, cutting by the cutting blade 602.

Alternatively, or in addition, the reach parameter may be configured to indicate an ability of the cutting blade 602 to reach, and thus, cut at least one the bone surface corresponding to one of the implant surface planes. The surgical computer 150 may then be configured to cause alignment of the cutting blade 602 using the determined the implant surface plane (e.g., implant surface plane P1 606a). It may also select a specific side of the cutting blade 602. The side selected by the surgical computer 150 may be configured to be proximate to the implant surface plane implant surface plane P1 606a. Alternatively, or in addition, the surgical computer 150 may select any side of the cutting blade 602 for the purposes of cutting the bone surface using the determined implant surface plane P1 606a. Once the cutting blade 602 is aligned to the specific implant plane and, optionally, a particular side of the cutting blade 602 is selected, the surgical computer 150 may be configured to trigger actuation of the cutting blade 602 to cut the bone surface using the selected side of the cutting blade 602.

In some examples, the surgical computer 150 may be configured to determine a reach parameter that may indicate that an ability of the cutting blade 602 to cut the bone surface using a plurality of implant surface planes (e.g., implant surface plane P1 606a and implant surface plane P2 606b) of the implant 604. In this case, the surgical computer 150 may be configured to identify a first implant surface plane (e.g., implant surface plane P1 606a) for alignment of the cutting blade 602. The surgical computer 150 may identify the implant surface plane P1 606a based on at least one of: a smallest distance from the reference point 612 on the cutting blade 602 to one or more transition edges of the implant surface plane, e.g., the transition edge 608a. Alternatively, or in addition, the surgical computer 150 may determine a smallest rotational angle of the cutting blade 602 when determining the specific implant surface plane. As can be understood, the surgical computer 150 may use any other factors and/or any combination of factors when identifying a particular implant surface plane for alignment.

Once that implant surface plane (e.g., implant surface plane P1 606a) is identified, the surgical computer 150 may be configured to align the cutting blade 602 using the identified implant surface plane. It may also, optionally, select a particular side of the cutting blade 602 that may be proximate to the identified implant surface plane. Once alignment of the cutting blade 602 and/or selection of the specific side of the cutting blade 602 for cutting is completed, the surgical computer 150 may be configured to trigger actuation of the cutting blade 602 to perform cutting of the bone surface using the selected side of the cutting blade 602.

FIG. 7 illustrates examples of normal distances between the cutting blade 602 and one or more transition edges 608, according to some examples of the current subject matter. As shown in FIG. 7, the surgical computer 150 may determine one or more normal distances N 702 (a, b, c, . . . ) that may exist between the cutting blade face 610 of the cutting blade 602 and the transition edges 608. For example, the surgical computer 150 may determine a normal distance N 1_2 702a to be between cutting blade face 610 and the transition edge 608a; a normal distance N 2 3 702b to be between cutting blade face 610 and the transition edge 608b; a normal distance N 3_4 702c to be between cutting blade face 610 and the transition edge 608c; etc. The surgical computer 150 may also determine that the normal distance N 1_2 702a is a negative normal distance with respect to the transition edge 608a indicating that the cutting blade 602 has already passed the transition edge 608a (as represented by an arrow pointing toward the cutting blade 602). Conversely, the surgical computer 150 may determine that normal distance N 2 3 702b and normal distance N 3_4 702c are positive normal distances with respect to transition edge 608b and transition edge 608c, respectively, indicating that that transition edge 608b and transition edge 608c have not been passed by the cutting blade 602 (as presented by arrows point away from the cutting blade 602).

In some examples, the surgical computer 150 may be configured to execute an automatic cut implant surface plane detection process using current position(s) and/or rotation parameters of the cutting blade 602. This may allow the surgeon to fluidly transition between necessary cuts on the bone without intermediate steps, which may be useful when trying to avoid soft tissue when moving between multiple implant surface planes.

As part of this process, the surgical computer 150 may be provided and/or may determine various information associated with the implant 604. This may include, for example, positions and normal distances (e.g., distances d1_2, d2_3, etc., distances 702, etc., as shown in FIGS. 6 and 7) of all implant surface planes, any surface meshes indicating exact dimensions of the implant surface planes (which may correspond to an imprint from the implant 604). Once the information is received by the surgical computer 150, the surgical computer 150 may be configured to determine the reach parameter by traversing over all provided information and solving kinematics using the current positions of the motor (e.g., motor 314) to determine if any implant surface planes are align-able to and within the reach of the cutting blade 602. In some examples, the surgical computer 150 may be configured to make this determination for both sides of the cutting blade 602. As discussed herein, the surgical computer 150 may determine one or more of the following results. One outcome of this determination may be that no implant surface planes are in reach (as indicated by the reach parameter), which may result in the surgical computer 150 preventing alignment and/or actuation of the cutting blade 602 to and/or of any plane. Another outcome may be that one implant surface plane is in reach (as indicated by the reach parameter), which may result in the surgical computer 150 causing the cutting blade 602 to align with that plane. Yet another outcome may be that multiple implant surface planes are in reach (as determined by the reach parameter). If multiple planes are within reach, the surgical computer 150 may cause the cutting blade 602 to align with the plane that may have the smallest linear distance to the reference point 612 of the cutting blade 602, and/or, alternatively, or in addition, the surgical computer 150 may be configured to compute a cost function of smallest linear distance and smallest rotational angle to determine specific plane. Once the surgical computer 150 made the above determination for each side of the cutting blade 602, the surgical computer 150 may select the side of the cutting blade 602 having the smallest distance as the active plane (i.e., which the cutting blade 602 will cut).

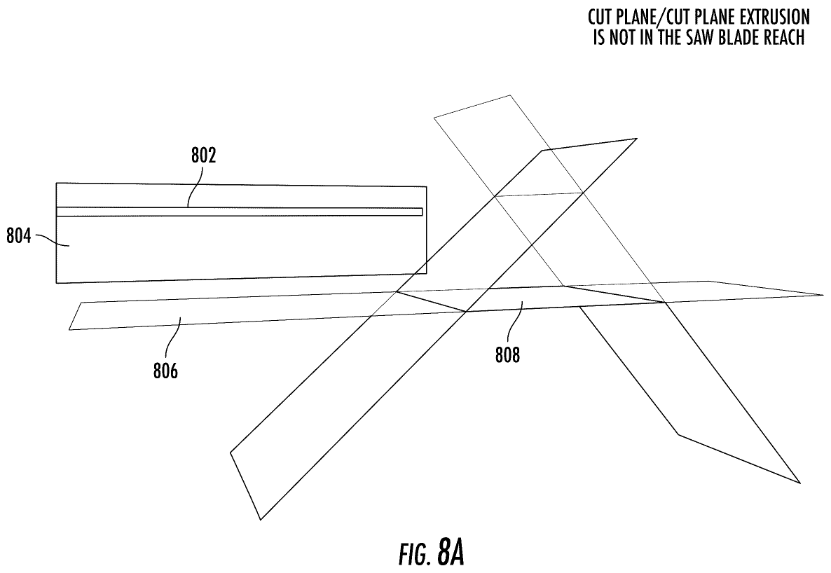

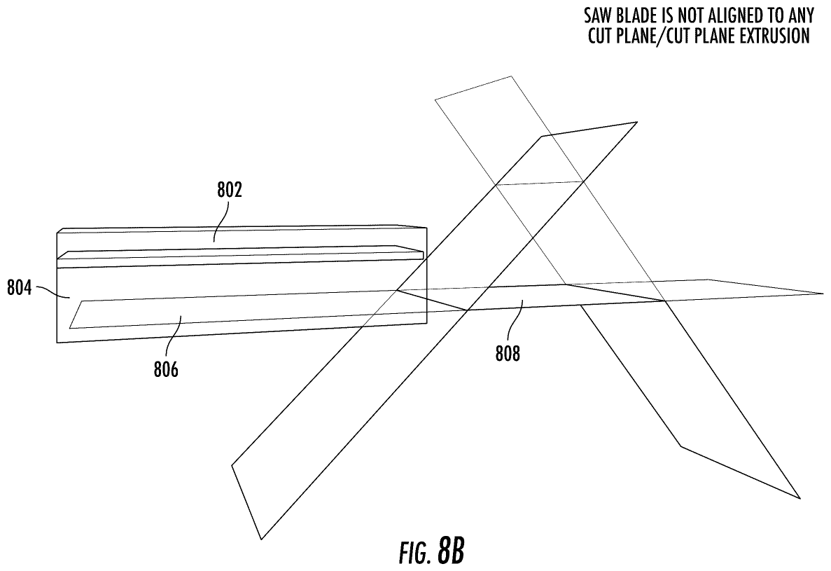

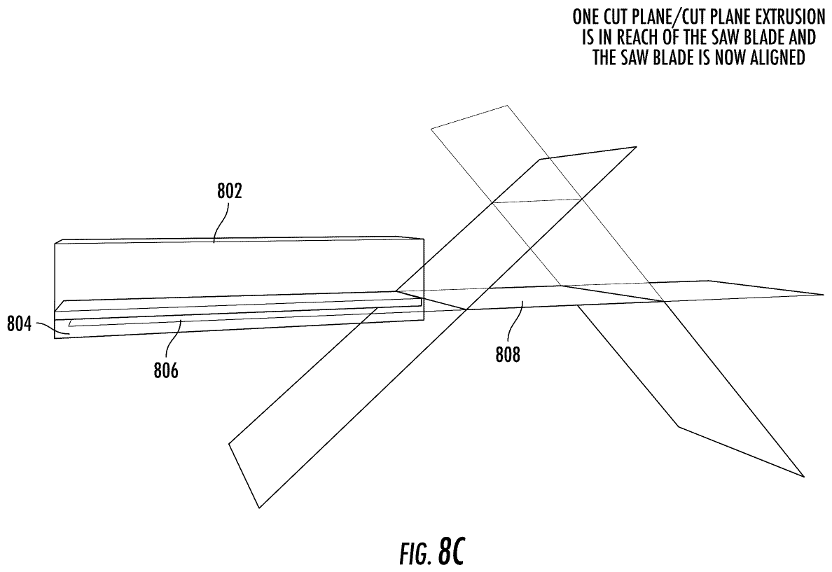

In some examples, the surgical computer 150 may perform a final check for any overcuts. After determining the current plane, the surgical computer 150 may again traverse all information (e.g., distances, dimensions, etc.) that it received, determined, and/or identified, while excluding the currently aligned plane, to determine if the cutting blade 602 intersects with any of the surface meshes. An intersection may indicate an overcut, thereby the surgical computer 150 may prevent actuation of the cutting blade 602. This is especially important for planes with concave angles, where controlling the plunging distance (which may be defined by a depth of cutting by the cutting blade 602) may be crucial (as shown in FIGS. 8A-C, 9A-B, and 10A-C).

FIG. 8A illustrates an example outcome of the determinations performed by the surgical computer 150 of the CASS 100, according to some examples of the current subject matter. In particular, FIG. 8A illustrates a cutting blade 802 (similar to cutting blade 602 shown in FIG. 6) having a cutting blade reach 804. As shown, none of the planes (e.g., cut plane extrusion 806 and/or cut plane 808) are within the cutting blade reach 804. As such, the surgical computer 150 may be configured to prevent actuation of the cutting blade 802.

FIG. 8B illustrates another example outcome of the determinations performed by the surgical computer 150, according to some examples of the current subject matter. In this scenario, the cutting blade 802 having the cutting blade reach 804 is within reach of the cut plane extrusion 806 and/or cut plane 808, and thus, the surgical computer 150 may cause the cutting blade 802 to attempt to align with one or more of the planes in accordance with the process discussed herein. As such, if alignment of the cutting blade 802 is achieved, the surgical computer 150 may be configured to trigger actuation of the cutting blade 802.

FIG. 8C illustrates another example outcome of the determinations performed by the surgical computer 150, according to some examples of the current subject matter. In this case, the cutting blade 802 having the cutting blade reach 804 is within reach of and is aligned to the cut plane extrusion 806 and/or cut plane 808, and thus, the surgical computer 150 may be configured to trigger actuation of the cutting blade 802.

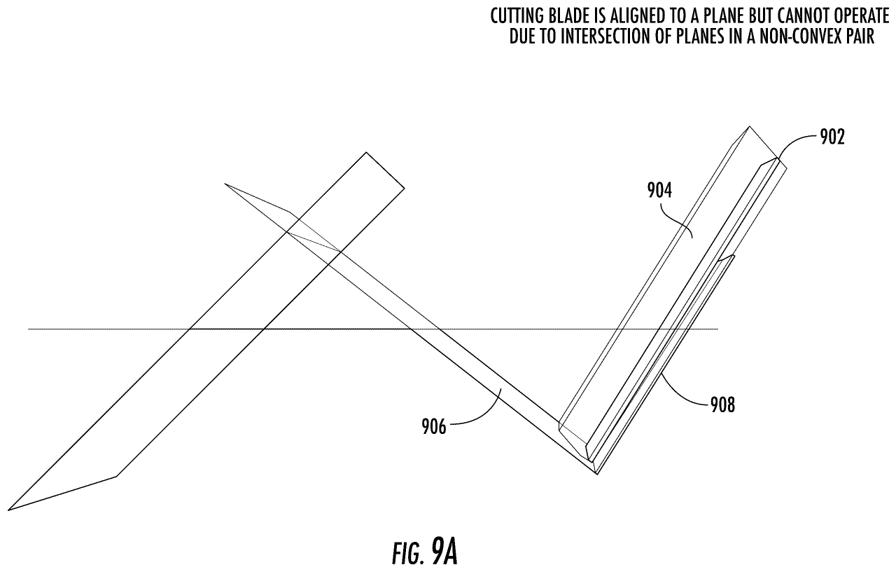

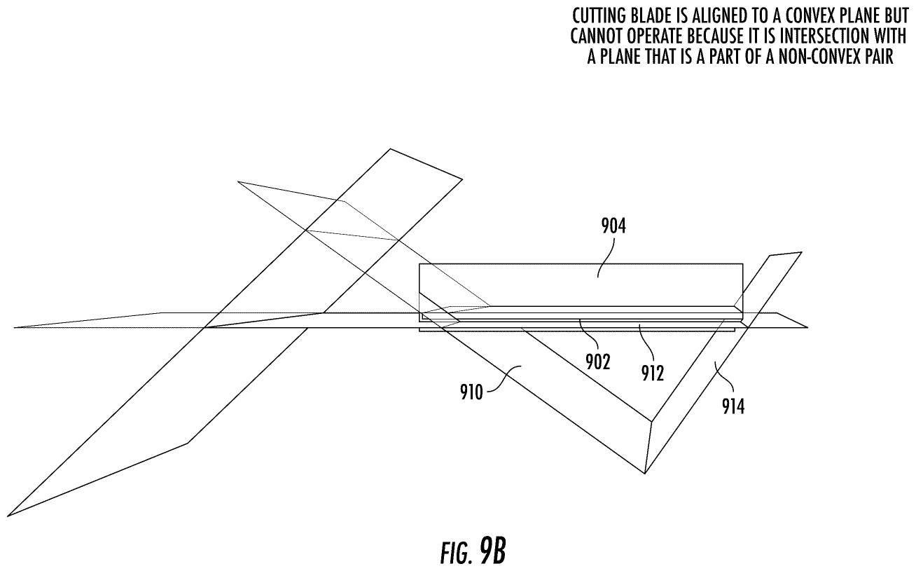

FIG. 9A illustrates another example outcome of the determinations performed by the surgical computer 150, according to some examples of the current subject matter. Here, one or more non-convex planes are being assessed by the surgical computer 150 to determine whether the cutting blade 902 having cutting blade reach 904 may be plunged for cutting of the bone. As shown in FIG. 9A, the cutting blade 902 cannot fully plunge due to the intersection of cut plane 906 and cut plane 908. As such, the surgical computer 150 may be configured to prevent actuation of the cutting blade 902.

FIG. 9B illustrates yet another example outcome of the determinations performed by the surgical computer 150, according to some examples of the current subject matter. In this case, the cutting blade 902 may be aligned to a convex cut plane 912, however, it is intersecting with a plane (e.g., cut plane 910 and/or cut plane 914) that is part of a non-convex pair. The saw blade is no longer able to plunge due to this intersection. Thus, the surgical computer 150 may be configured to prevent actuation of the cutting blade 902.

A similar approach may be used to cut curved surfaces of bones corresponding to implant surface with the cutting blade. In this case, instead of modelling each plane using a reference point on the cutting blade, a normal distance, and surface the curved implant surface, the surgical computer 150 may use a spline equation to perform the modeling.

The surgical computer 150 may pre-calculate the spline equation per implant by sampling N points from a curve and feeding these points into a spline fitting algorithm. In the case where the curvature cannot be solved with a low error, the surface may be arbitrarily split up into multiple splines or small surface planes. The surgical computer 150 may perform assessment of this “set” of splines in a similar fashion to the one discussed herein. In particular, the surgical computer 150 may iterate over the splines to determine a particular one to use.

In some examples, the surgical computer 150 may generate an orthogonal projection from the center of the cutting blade face both on the upward and downward facing side. The surgical computer 150 may then check the projection for any intersection(s) against one or more spline(s). Depending on the curvature of the implant, the surgical computer 150 may determine one or more of the following. In one scenario, no intersection may be determined by the surgical computer 150, thereby causing the surgical computer 150 to prevent actuation of the cutting blade. IN another scenario, the surgical computer 150 may determine that a single intersection exists, in which case the intersection point may be used for further determinations (in accordance with the methodology discussed herein. In yet another scenario, the surgical computer 150 may determine that there may be two or more intersections, in which case the intersection that has the closest distance to the cutting blade may be used for further computation (this case may be applicable in non-convex geometries). Once intersection point is determined, the surgical computer 150 may then convert this point and its normal distance into information that may be used by the surgical computer 150 to determine whether actuation of the cutting blade may be performed. The surgical computer 150 may then check to determine whether this point is reachable within the current limits of the cutting blade and if so, the surgical computer 150 may then attempt to align the cutting blade. The surgical computer 150 may also verify that the cutting blade is not intersecting with the spline (in case of nonconvex curvature). This may be done using a plane to surface intersection check.

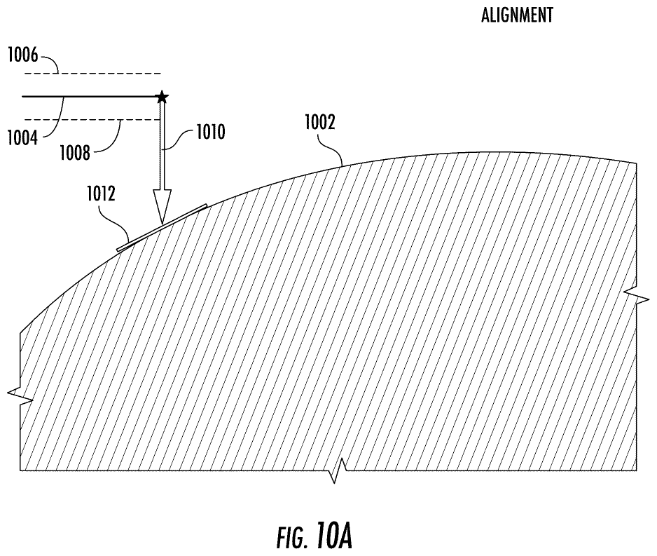

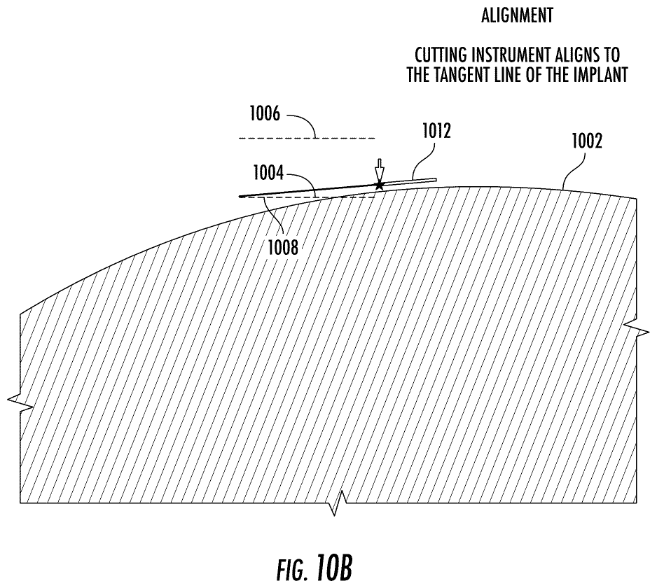

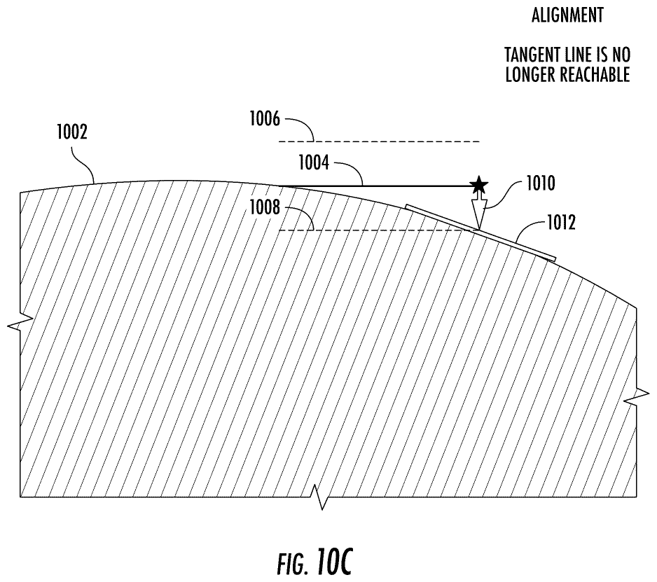

FIG. 10A illustrates an example of an alignment determination performed by the surgical computer 150, according to some examples of the current subject matter. The surgical computer 150 may analyze the parameters of a curved surface 1002 in relation to the cutting blade 1004 having an upper bound of linear actuator 1006 and a lower bound of linear actuator 1008. To do that it may determine an orthogonal projection 1010 to the tangent line 1012 on the curved surface 1002. As shown in FIG. 10B, the surgical computer 150 may determine that the cutting blade 1004 is aligned to the tangent line 1012 of the implant, and may, then determine whether or not to actuate the cutting blade 1004. Alternatively, as shown in FIG. 10C, the surgical computer 150 may determine that the cutting blade 1004 is not aligned to the tangent line 1012 by determining that the tangent line 1012 is no longer reachable by the cutting blade 1004, thereby preventing actuation of the cutting blade 1004.

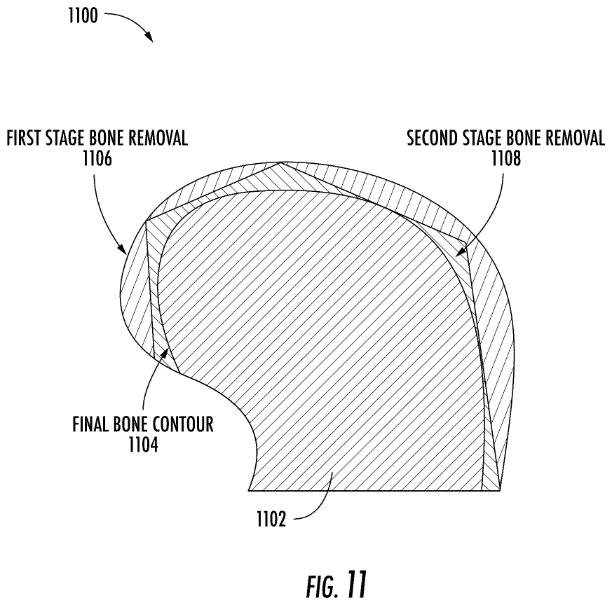

FIG. 11 illustrates an example of a multi-stage bone cutting process 1100, according to some examples of the current subject matter. The multi-stage bone cutting process of a bone 1102 may be performed by the CASS 100, and in particular using surgical computer 150. As discussed herein, the process 1100 may be helpful when various bone dimensions, surgical instrument limitation parameters, and/or any other constraints, etc. may limit operational capabilities of the cutting blade (e.g., rotation of the cutting blade to a predetermined degree preventing the cutting blade to reach a particular surface (e.g., a curved surface).

Using the process 1100, the CASS 100 may be configured to perform a predetermined number of planar bone cuts or “rough cuts”, e.g., first stage bone removal 1106, to remove tissue along one or more planes, as shown in FIG. 11. The first stage bone removal 1106 may be performed in accordance with the techniques described herein. As can be understood one or more first stage bone removal 1106 may be performed (and/or repeated).

Once first stage bone removal 1106, more finer cuts, e.g., second stage bone removal 1108, along a predetermined final bone contour 1104 may be performed. The final bone contour 1104 may be defined by one or more implant surfaces and/or any other parameters. Similarly, the second stage bone removal 1108 using the processes described herein. One or more such second stage bone removal 1108 may be performed (and/or repeated). The multi-stage bone cutting process 1100 may reduce cutting blade's inability to reach certain surfaces and allow the cutting blade to more precisely cut the same.

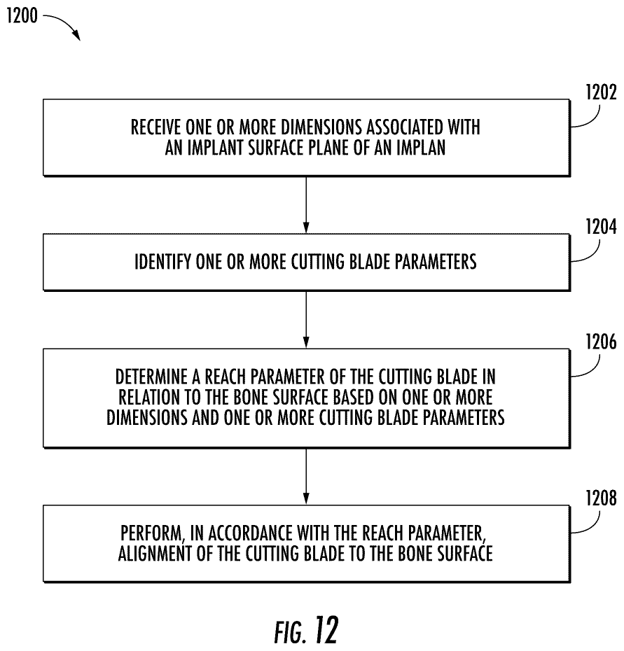

FIG. 12 illustrates an example process 1200 for positioning and operating a surgical instrument, according to some examples of the current subject matter. The process 1200 may be executed using surgical computer 150 of the CASS 100 to determine whether or not a cutting blade 308 of the CASS 100 can be actuated to perform cutting of bone tissue in accordance with and/or as defined by one or more surface planes of an implant 604. The implant 604 may have a single surface plane and/or multiple surface planes.

At 1202, surgical computer 150 may be configured to receive one or more dimensions associated with an implant surface plane (e.g., surface plane(s) 606) of an implant (e.g., implant 604). The implant surface plane may be defined by at least one transition edge (e.g., transition edge(s) 608) separating the implant surface plane from another implant surface plane of the implant. The implant may be configured for implantation into a bone of a patient.

At 1204, the surgical computer 150 may determine and/or identify one or more surgical instrument limitation parameters and determine and/or identify one or more cutting blade parameters. The surgical instrument limitation parameters may define various mechanical, electrical, and/or electro-mechanical limitations of the surgical instrument, including the cutting blade (e.g., rotational, translational, etc. limitations). The cutting blade parameters may include a reference distance representative of a distance from a reference location (e.g., reference point 612) on the cutting blade to the at least one transition edge, a normal distance (e.g., normal distance(s) 702) from at least one transition edge to a surface of the cutting blade, and a direction of cutting of the cutting blade.

At 1206, the surgical computer 150 may determine a reach parameter of the cutting blade in relation to the bone surface based on one or more dimensions, one or more surgical instrument limitation parameters, and one or more cutting blade parameters. The reach parameter may indicate an ability of the cutting blade to cut the bone surface using one or more dimensions and one or more cutting blade parameters.

At 1208, the surgical computer 150 may perform, in accordance with the reach parameter, alignment of the cutting blade to the bone surface. In some examples, alignment may be automatically performed and/or attempted to be performed once the reach parameter is determined. The alignment of the cutting blade to the bone surface may be performed using one or more discrete planes and/or a surface subdivided into discrete planes (e.g., the implant surface plane, another implant surface plane, etc.). Alternatively, or in addition, alignment may be performed using a tangent line of the implant surface (e.g., closest tangent line). The surfaces may be splined and/or geometrically/analytically defined. Once alignment is achieved, a determination may be made whether or not to actuate the cutting blade in accordance with the reach parameter. One or more portions of the bone may then be removed based on the alignment (using any of the of above alignments, for example).

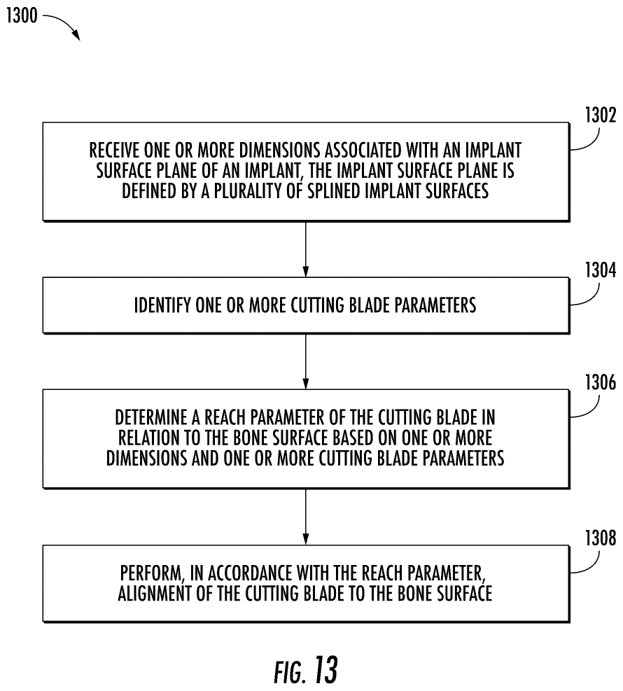

FIG. 13 illustrates another example process 1300 for positioning and operating a surgical instrument, according to some examples of the current subject matter. The process 1300 may also be executed using surgical computer 150 of the CASS 100 to determine whether or not a cutting blade 308 of the CASS 100 can be actuated to perform cutting of bone tissue in accordance with and/or as defined by one or more surface planes of the implant 604. This process may be executed for implants having complex surfaces.

At 1302, the surgical computer 150 may receive one or more dimensions associated with an implant surface plane of an implant (e.g., implant 604). The implant surface plane may be defined by a plurality of splined implant surfaces. Each splined implant surface may include at least one transition edge separating one splined implant surface from another splined implant surface in the plurality of splined implant surfaces. The implant may be configured for implantation into a bone of a patient.

At 1304, the surgical computer 150 may determine and/or identify one or more surgical instrument limitation parameters and determining and/or identify one or more cutting blade parameters including a reference distance representative of a distance from a reference location on the cutting blade to at least one transition edge of at least one splined implant surface in the plurality of splined surfaces, a normal distance from at least one transition edge to a surface of the cutting blade, and a direction of cutting of the cutting blade. The reference distance may be determined based on an orthogonal projection to the implant surface plane.

At 1306, the surgical computer 150 may determine a reach parameter of the cutting blade in relation to the bone surface based on one or more dimensions, one or more surgical instrument limitation parameters, and one or more cutting blade parameters. The reach parameter may indicate an ability of the cutting blade to cut the bone surface using one or more dimensions and one or more cutting blade parameters.

At 1308, the surgical computer 150 may perform, in accordance with the reach parameter, alignment of the cutting blade to the bone surface and/or determine whether to actuate the cutting blade in accordance with the reach parameter. One or more portions of the bone may then be removed based on the alignment. In some examples, alignment may be automatically performed and/or attempted to be performed once the reach parameter is determined. The alignment may be performed using one or more discrete planes and/or a surface subdivided into discrete planes, using a tangent line of the implant surface (e.g., closest tangent line). The surfaces may be splined and/or geometrically/analytically defined.

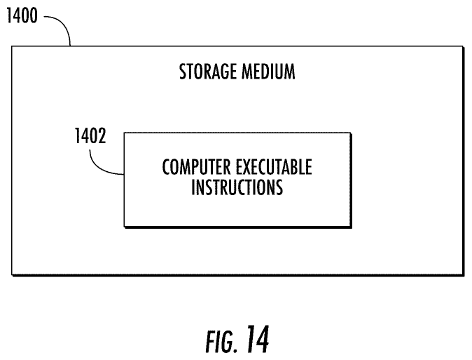

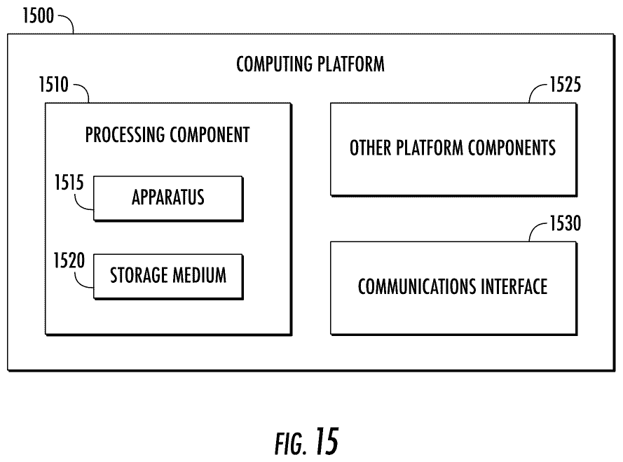

FIG. 14 and FIG. 15 illustrate example implementations of a storage medium and computing platform for an orthopedic surgical instrument or a surgical system in accordance with one or more features of the present disclosure. FIG. 14 illustrates an example of a storage medium 1400 to store system logic. Storage medium 1400 may include an article of manufacture. In some examples, storage medium 1400 may include any non-transitory computer readable medium or machine-readable medium, such as an optical, magnetic or semiconductor storage. Storage medium 1400 may store various types of computer executable instructions 1402, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 15 illustrates an example computing platform 1500. In some examples, as shown in FIG. 15, the computing platform 1500 may include a processing component 1510, other platform components 1525 or a communications interface 1530. According to some examples, computing platform 1500 may be implemented in a computing device such as a server in a system such as a data center or server farm that supports a manager or controller for managing configurable computing resources as mentioned above. Further, the communications interface 1530 may include a wake-up radio (WUR) and may be capable of waking up a main radio of the computing platform 1500.

According to some examples, processing component 1510 may execute processing operations or logic for apparatus 1515 described herein such as the surgical computer 150. Processing component 1510 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 1520, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 1525 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 1530 may include logic and/or features to support a communication interface. For these examples, communications interface 1530 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 1500 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 1500 described herein, may be included or omitted in various implementations of computing platform 1500, as suitably desired.

The components and features of computing platform 1500 may be implemented using any combination of discrete circuitry, ASICs, logic gates and/or single chip architectures. Further, the features of computing platform 1500 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the exemplary computing platform 1500 shown in the block diagram of FIG. 15 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in implementations.

One or more features of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

The foregoing description has broad application. While the present disclosure refers to certain implementations, numerous modifications, alterations, and changes to the described implementations are possible without departing from the sphere and scope of the present disclosure, as defined in the appended claim(s). Accordingly, it is intended that the present disclosure is not limited to the described implementations. Rather these implementations should be considered as illustrative and not restrictive in character. All changes and modifications that come within the spirit of the current subject matter are to be considered within the scope of the disclosure. The present disclosure should be given the full scope defined by the language of the following claims, and equivalents thereof. The discussion of any implementation is meant only to be explanatory and is not intended to suggest that the scope of the disclosure, including the claims, is limited to these implementations. In other words, while illustrative implementations of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counterclockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular implementations. Such terms are not generally limiting to the scope of the claims made herein. Any implementation or feature of any section, portion, or any other component shown or particularly described in relation to various implementations of similar sections, portions, or components herein may be interchangeably applied to any other similar implementation or feature shown or described herein.

It should be understood that, as described herein, an “implementation” and/or “examples” (terms used interchangeably herein) (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated implementations are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one implementation” of the present disclosure are not intended to be interpreted as excluding the existence of additional implementations that also incorporate the recited features.

In addition, it will be appreciated that while the Figures may show one or more implementations of concepts or features together in a single implementation of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one implementation may be used separately, or with another implementation to yield a still further implementation. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “includes” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” may be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more implementations or configurations for the purpose of streamlining the disclosure. However, it should be understood that various features of the certain implementations or configurations of the disclosure may be combined in alternate implementations or configurations. Moreover, the following claims are hereby incorporated into this detailed description by this reference, with each claim standing on its own as a separate implementation of the present disclosure.

What is claimed is:

1. A computer-implemented method, comprising:
receiving, using at least one processor of a surgical instrument, one or more dimensions associated with an implant surface plane of an implant, the implant surface plane is defined by at least one transition edge separating the implant surface plane from another implant surface plane of the implant, the implant is configured for implantation into a bone of a patient, the surgical instrument includes a cutting blade for cutting a bone surface in the bone corresponding to the implant surface plane;
identifying, using the at least one processor, one or more surgical instrument limitation parameters and one or more cutting blade parameters including a reference distance representative of a distance from a reference location on the cutting blade to the at least one transition edge, a normal distance from the at least one transition edge to a surface of the cutting blade, and a direction of cutting of the cutting blade;
determining, using the at least one processor, a reach parameter of the cutting blade in relation to the bone surface based on the one or more dimensions, the surgical instrument limitation parameters, and the one or more cutting blade parameters, the reach parameter indicating an ability of the cutting blade to cut the bone surface using the one or more dimensions, the one or more surgical instrument limitation parameters, and the one or more cutting blade parameters; and
performing, in accordance with the reach parameter, alignment of the cutting blade to the bone surface.

2. The method of claim 1, wherein performing includes automatically performing, in accordance with the reach parameter, the alignment of the cutting blade.

3. The method of claim 1, wherein the alignment of the cutting blade to the bone surface is performed using at least one of: the implant surface plane, the another implant surface plane, or any combination thereof.

4. The method of claim 1, wherein the alignment of the cutting blade to the bone surface is performed using a tangent line of at least one of: the implant surface plane, the another implant surface plane, or any combination thereof, wherein the bone surface is at least one of: a splined bone surface, an analytically defined surface, or any combination thereof.

5. The method of claim 1, further comprising actuating, using the at least one processor, the cutting blade in accordance with the reach parameter and based on the alignment.

6. The method of claim 5, further comprising removing at least a portion in a plurality of portions of the bone surface using the alignment of cutting blade.

7. The method of claim 5, wherein the actuating, upon the reach parameter indicating an inability of the cutting blade to cut the bone surface using the implant surface plane, including preventing cutting by the cutting blade.

8. The method of claim 5, wherein the actuating, upon the reach parameter indicating an ability of the cutting blade to cut the bone surface using the implant surface plane, including
aligning the cutting blade using the implant surface plane;
selecting a side of the cutting blade proximate to the implant surface plane; and
actuating the cutting blade for cutting the bone surface using the selected side.

9. The method of claim 5, wherein the actuating, upon the reach parameter indicating an ability of the cutting blade to cut the bone surface using a plurality of implant surface planes of the implant, including
identifying a first implant surface plane in the plurality of implant surface planes based on at least one of: a smallest distance representative of a distance from the reference location on the cutting blade to at least one transition edge of the first implant surface plane, a smallest rotational angle of the cutting blade, or any combination thereof;
aligning the cutting blade using the first implant surface plane;
selecting a side of the cutting blade proximate to the first implant surface plane; and
actuating the cutting blade for cutting the bone surface using the selected side.

10. The method of claim 1, wherein the surgical instrument is a surgical saw.

11. The method of claim 1, wherein the one or more dimensions include one or more plane coordinates associated with the at least one transition edge between at least one implant surface plane and at least another implant surface plane in a plurality of implant surface planes of the implant.

12. The method of claim 11, wherein the reach parameter is determined based on a combination of a minimum absolute distance between the distal end of the cutting blade and the one or more coordinates associated with the transition edge and a negative normal distance, the negative normal distance is determined using a difference between the normal distance and the reference distance and the negative direction of cutting of the cutting blade.

13. A computer-implemented method, comprising:
receiving, using at least one processor of a surgical instrument, one or more dimensions associated with an implant surface plane of an implant, the implant surface plane is defined by a plurality of splined implant surfaces, each splined implant surface includes at least one transition edge separating one splined implant surface from another splined implant surface in the plurality of splined implant surfaces, the implant is configured for implantation into a bone of a patient, the surgical instrument includes a cutting blade for cutting a bone surface in the bone corresponding to at least one splined implant surface in the plurality of splined implant surfaces;
identifying, using the at least one processor, one or more surgical instrument limitation parameters, one or more cutting blade parameters including a reference distance representative of a distance from a reference location on the cutting blade to the at least one transition edge of at least one splined implant surface in the plurality of splined surfaces, a normal distance from the at least one transition edge to a surface of the cutting blade, and a direction of cutting of the cutting blade, wherein the reference distance is determined based on an orthogonal projection to the implant surface plane;
determining, using the at least one processor, a reach parameter of the cutting blade in relation to the bone surface based on the one or more dimensions, the one or more surgical instrument limitation parameters, and the one or more cutting blade parameters, the reach parameter indicating an ability of the cutting blade to cut the bone surface using the one or more dimensions, the surgical instrument limitation parameters, and the one or more cutting blade parameters; and
performing, in accordance with the reach parameter, alignment of the cutting blade to the bone surface.

14. The method of claim 13, wherein performing includes automatically performing, in accordance with the reach parameter, the alignment of the cutting blade.

15. The method of claim 13, wherein the alignment of the cutting blade to the bone surface is performed using at least one of: the implant surface plane, the another implant surface plane, or any combination thereof.

16. The method of claim 13, wherein the alignment of the cutting blade to the bone surface is performed using a tangent line of at least one of: the implant surface plane, the another implant surface plane, or any combination thereof, wherein the bone surface is at least one of: a splined bone surface, an analytically defined surface, or any combination thereof.

17. The method of claim 13, further comprising actuating, using the at least one processor, the cutting blade in accordance with the reach parameter.

18. The method of claim 17, further comprising removing at least a portion in a plurality of portions of the bone surface using the alignment of cutting blade.

19. The method of claim 17, wherein the reach parameter indicates an inability of the cutting blade to cut the bone surface using the implant surface plane upon the orthogonal projection not intersecting any splined implant surfaces in the plurality of splined implant surfaces, wherein the actuating includes preventing cutting by the cutting blade.

20. The method of claim 17, wherein the reach parameter indicates an ability of the cutting blade to cut the bone surface using at least one splined implant surface in the plurality of splined implant surfaces upon the orthogonal projection intersecting the at least one splined implant surface, wherein the actuating includes
aligning the cutting blade using the splined implant surface;
selecting a side of the cutting blade proximate to the splined implant surface; and
actuating the cutting blade for cutting the bone surface using the selected side.

21. The method of claim 17, wherein the reach parameter indicates an ability of the cutting blade to cut the bone surface using two or more splined implant surfaces in the plurality of splined implant surfaces upon the orthogonal projection intersecting the two or more splined implant surfaces, wherein the actuating includes
identifying a first splined implant surface in the two or more splined implant surfaces based on at least one of: a smallest distance representative of a distance from the reference location on the cutting blade to at least one transition edge of the first splined implant surface, a smallest rotational angle of the cutting blade, or any combination thereof;
aligning the cutting blade using the first splined implant surface;
selecting a side of the cutting blade proximate to the first splined implant surface; and
actuating the cutting blade for cutting the bone surface using the selected side.

22. A surgical system, comprising:
a surgical instrument having a cutting blade for cutting a bone surface in a bone of a patient corresponding to an implant surface plane of an implant, the implant surface plane is defined by at least one transition edge separating the implant surface plane from another implant surface plane of the implant, the implant is configured for implantation into the bone;
at least one processor; and
at least one memory storing instructions that, when executed by the at least one processor, cause the at least one processor to:
receive one or more dimensions associated with the implant surface plane;
identify one or more surgical instrument limitation parameters and one or more cutting blade parameters including a reference distance representative of a distance from a reference location on the cutting blade to the at least one transition edge, a normal distance from the at least one transition edge to a surface of the cutting blade, and a direction of cutting of the cutting blade;
determine a reach parameter of the cutting blade in relation to the bone surface based on the one or more dimensions, the one or more surgical instrument limitation parameters, and the one or more cutting blade parameters, the reach parameter indicating an ability of the cutting blade to cut the bone surface using the one or more dimensions, the one or more surgical instrument limitation parameters, and the one or more cutting blade parameters; and
perform, in accordance with the reach parameter, alignment of the cutting blade to the bone surface.
